(19) United States
(12) Patent Application Publication
(10) Pub. No.: US 2024/0383854 A1
(43) Pub. Date: Nov. 21, 2024

(54) PROCESS FOR THE PREPARATION OF CHLOROALKYL SUBSTITUTED CYCLIC AMINES

(71) Applicant: HOVIONE SCIENTIA, County Cork (IE)

(72) Inventors: Marianna KATZ, Lisboa (PT); Nuno TORRES, Lisboa (PT); Luis SOBRAL, Montijo (PT); Rafael ANTUNES, Setubal (PT)

(21) Appl. No.: 18/688,885

(22) PCT Filed: Sep. 20, 2021
(86) PCT No.: PCT/EP2021/075830

§ 371 Date: Mar. 4, 2024

(30) Foreign Application Priority Data
Sep. 3, 2021 (PT) 117440

(51) Int. Cl. C07D 211/62; C07D 241/04; C07D 265/00; C07D 267/00; C07D 295/067

(52) U.S. Cl. CPC C07D 211/62; C07D 241/04; C07D 265/00; C07D 267/00; C07D 295/067

(57) ABSTRACT

A process for the preparation of a compound of formula (II) where Et=ethyl; Bn=benzyl and Ph=phenyl; comprises the step of reacting a cyclic amine with an alkylating agent to form a compound of formula II, wherein the process is solvent-free. The chloroalkyl N-substituted cyclic amines of formula II may be used in the preparation of active pharmaceutical ingredients or intermediates therefor comprising these moieties in their molecular structure.

The present invention relates generally to a process for the preparation of substituted cyclic amines, especially but not exclusively 1-(2-chlorethyl) and 1-(3-chloropropyl) substituted cyclic amines, and in particular wherein the cyclic amine is piperidine, or piperazine, or morpholine; or pyrrolidine or hexamethyleneimine. In particular, the process comprises reacting a cyclic secondary amine with a bifunctional alkylating agent in the presence of an organic base in batch or continuous flow mode, under solvent-free conditions, to form the respective chloroalkyl substituted cyclic amine.

### BACKGROUND OF THE INVENTION

Several organic compounds, such as pharmaceutical active ingredients (APIs) and agrochemicals possess N-alkylated piperidine, piperazine or morpholine moieties in their structure, as in, for example, umeclidinium bromide, ziprasidone, risperidone, trifluoperazine, trazodone, gefitinib, doxapram, domperidone, cetiedil, nabazenil, setastine, fedratinib, pitolisant, tridemorph, silylpropylamine derivatives, 1H-1,2,4-triazole derivatives and N-substituted 3-aryl-pyrrolidine derivatives. The synthesis of these organic compounds usually requires the use of an N-chloroalkylated ring with a proper substitution pattern (Scheme 1).

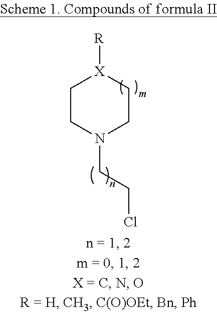

The alkylation of these nitrogen rings is widely reported (Scheme 2, and several synthetic strategies have been performed to overcome the poor yields observed due to side products formation, particularly the corresponding dimer, which usually requires product isolation by chromatography.

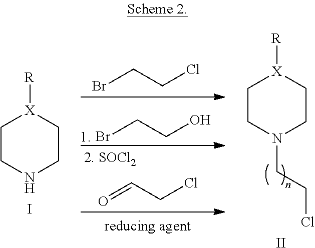

The most common approach to prepare chloroalkyl substituted compounds of formula II comprises the reaction of 1-bromo-2-chloroethane or 1-bromo-3-chloropropane with the corresponding cyclic amine in the presence of a base, traditionally potassium carbonate, in a solvent such as acetone or acetonitrile (Scheme 3). However, yields are often low and unwanted dimers often form.

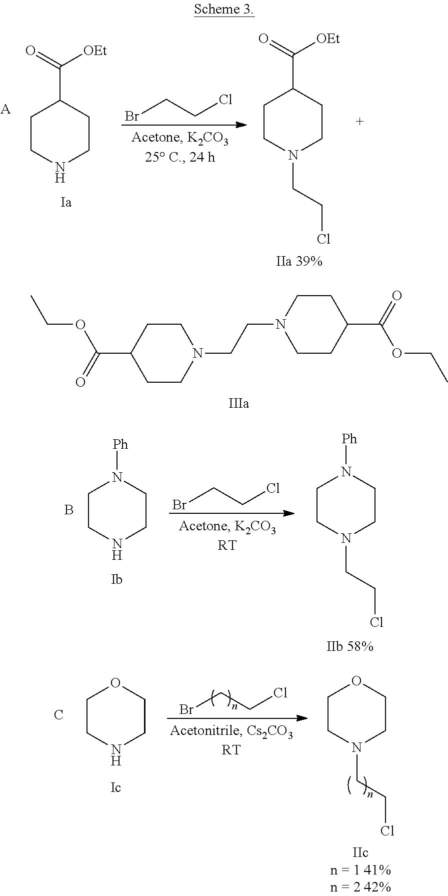

Patent application WO2005/104745 reports the preparation of 1-(2-chloroethyl)piperidine-4-carboxylate (IIa) by reacting 1-bromo-2-chloroethane with ethyl isonipecotate (Ia) in the presence of potassium carbonate in acetone (Scheme 3, A). However, compound IIa was attained in very low yields (39%) due to formation of the dimeric side product—diethyl 1,1′-(ethane-1,2-diyl)bis(piperidine-4-carboxylate) (IIIa), which was separated from compound IIa by chromatography.

The same conditions were employed to prepare several haloalkyl-4-phenylpiperazines with yields ranging from 45% to 61% (Bioorganic Med. Chem. 2016, 24, 2137), particularly 1-(2-chloroethyl)-4-phenylpiperazine (IIb) which was obtained from 1-phenylpiperazine in 58% yield after chromatographic purification (Scheme 3, B).

1-(2-Chloroethyl)- or 1-(3-chloropropyl)-morpholine (IIc) were prepared by reacting 1-bromo-2-chloroethane or 1-bromo-3-chloropropane with morpholine in the presence of cesium carbonate in acetonitrile (Scheme 3, C). Products IIc (n=1 and n=2) were isolated and obtained in 41% and 42% yield, respectively (RSC Advances 2015, 5, 103172). The same authors also report the preparation of 1-(3-chloropropyl)-4-methylpiperazine in 38% employing the same procedure.

More recently, patent application WO2018/087561 claimed that the method could be improved by using an organic base in acetone, yielding IIa with 66% yield and a maximum of 14% for IIIa.

Alternative conditions were described in Bioorganic Med. Chem. 2000, 8, 533 and Eur. J. Med. Chem. 1995, 30, 77 to prepare 1-(2-chloroethyl)-4-phenylpiperazine and 1-(2-chloroethyl)-4-methylpiperazine, respectively. The compounds were prepared in moderate yields by reacting 1-phenylpiperazine and 1-methylpiperazine with 1-bromo-2-chloroethane in toluene under reflux in the absence of base. Both publications report the formation of the corresponding dimeric side products.

In order to overcome the dimerization issue and the consequent low yields, a two-step alternative process was adopted for the preparation of compounds of formula II: a) reaction of the nitrogen ring with 2-bromoethanol or 2-chloroethanol to attain the ethyl alcohol intermediates IV followed by b) reaction of IV with thionyl chloride in a suitable solvent to give the corresponding chloroalkyl cyclic amines II (Scheme 4).

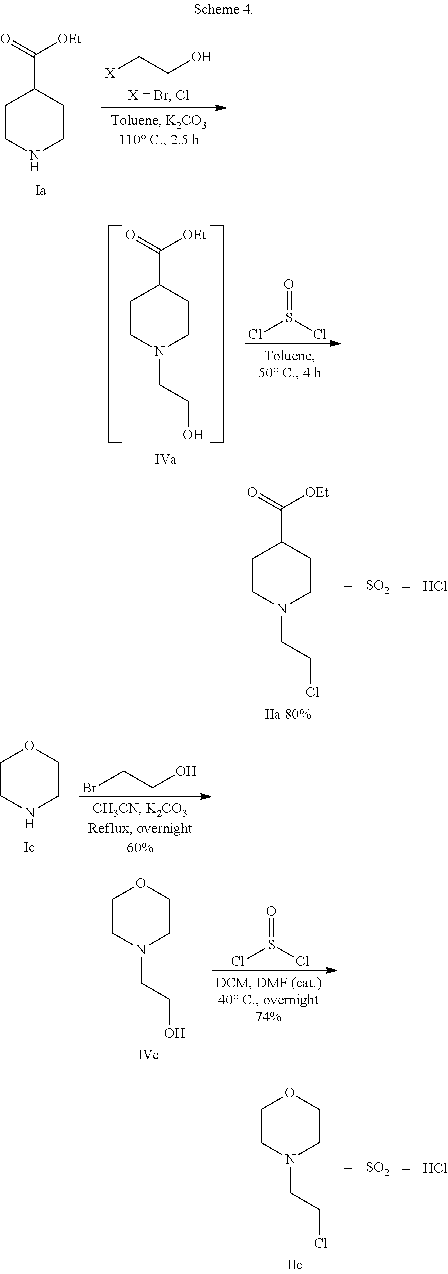

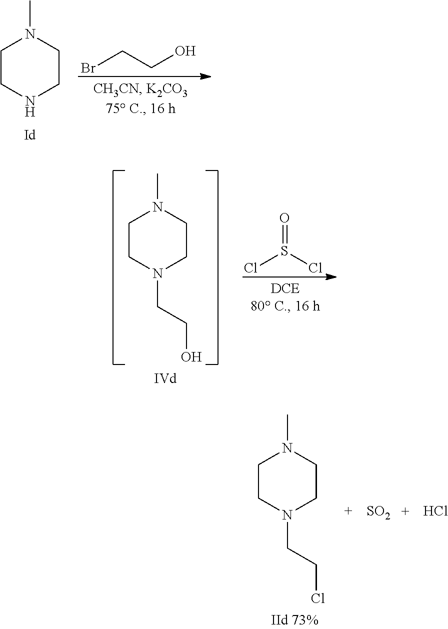

Patent application WO2014/027045 describes the preparation of a compound of formula IIa, wherein the first step comprises the reaction of Ia with 2-bromoethanol or 2-chloroethanol in the presence of potassium carbonate in toluene to form IVa. After aqueous work-up, IVa was reacted with thionyl chloride to obtain IIa in 80% yield (Scheme 4, A). An identical approach was reported in CN107200734. Alternatively, the first step can be carried out using triethylamine, in order to prepare 4-(2-chloroethyl)morpholine in 68% overall yield (ChemMedChem 2012, 7, 777). Patent application WO2017149518 describes the reaction of morpholine with 2-bromoethanol in the presence of potassium carbonate in acetonitrile to give IVb with 60% yield after isolation. The later was reacted with thionyl chloride in DCM to give IIb in 74% (Scheme 4, B).

Patent application WO2016/044666 reports the reaction of 1-methylpiperazine with 2-bromoethanol in the presence of potassium carbonate in acetonitrile, followed by reaction of IVc with SOCl₂ in 1,2-dichloroethane to obtain 1-(2-chloroethyl)-4-methylpiperazine (IId) in 73% (Scheme 4, C).

1-(2-Chloroethyl)-4-benzylpiperidine was also prepared under the same conditions from 4-benzylpiperidine (Bioorganic Med. Chem. Lett. 2000, 10, 527). The yields are not given for this compound but are stated yields above 60% for the transformation.

As alternative to the use of 2-bromoethanol or 2-chloroethanol, CN107935917 describes the use of oxirane in the first step to attain intermediate IVa (Scheme 5).

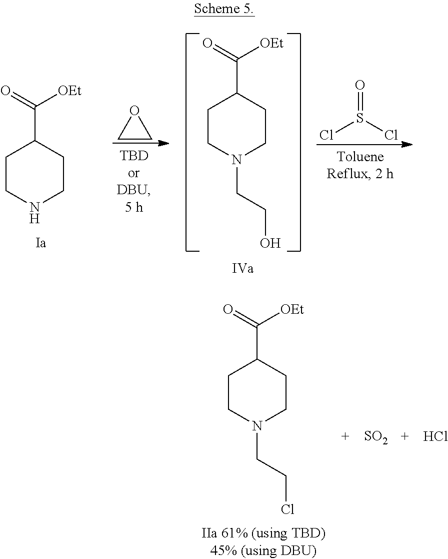

There is no doubt that such synthetic alternatives can lead to better yields, but the need for two reaction steps instead of one, is not the best solution for industrial application. Additionally, the use of high temperatures and a highly corrosive and toxic reagent that produces environmentally unfriendly SOₓ by-products is also not advisable for an industrial process.

An alternative strategy that avoids the use of thionyl chloride was described in patent U.S. Pat. No. 4,202,978, which claims a two-step process for the preparation of compound IIb: a) reaction of Ib with ethylene oxide to attain IVb followed by b) reaction of IVb with mesyl chloride to form IIb (Scheme 6). However, the yields were not reported.

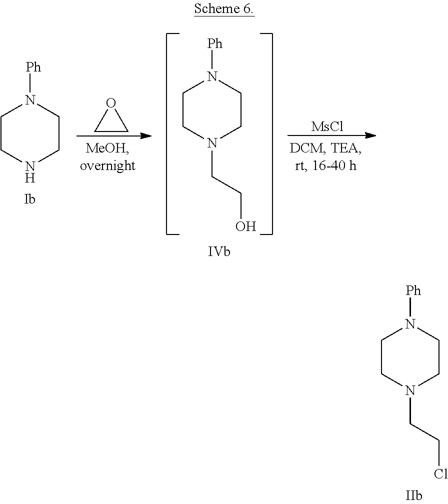

Patent application WO2016/071792 comprises a reductive amination of compound Ia with chloroacetaldehyde in a mixture of methanol/acetic acid using sodium cyanoborohydride as the reducing agent, yielding IIa in 90% yield (Scheme 7, A). Although leading to better yields, the synthesis requires the use of methanolic-aqueous acidic solutions, which can degrade the ester moiety.

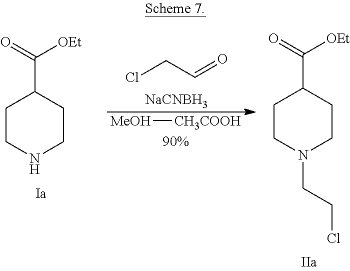

The same approach was described in Chem. Pharm. Bull. 1997, 45, 996 for morpholine and 1-methylpiperazine, which were reacted with chloroacetaldehyde in a mixture of DCM/acetic acid in the presence of sodium triacetoxyborohydride to yield compound IIb and IIc. The yields were not described.

Despite the reported methods, we have appreciated that there is in fact still a need to develop a more efficient procedure to attain these important N-chloroalkylated intermediates in high yield, with reduced impurities and in kilogram scale. Having appreciated this, the present inventors have now devised such a process—one which reduces or eliminates one or more of the problems reported above with the known processes.

### SUMMARY OF THE INVENTION

According to one aspect of the present invention, there is provided a process for the preparation of a compound of formula II:

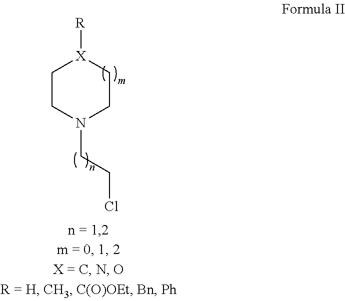

where Et=ethyl; Bn=benzyl and Ph=phenyl; comprising the step of reacting a cyclic amine with an alkylating agent to form a compound of formula II, wherein the process is solvent-free.

The alkylating agent is preferably a haloalkane compound, and is preferably a bifunctional alkylating agent, such as a bifunctional haloalkane. A suitable haloalkane compound is preferably an unsaturated straight chain alkane, preferably with 2, 3 or 4 carbon atoms. Typically it will be substituted with two halogen atoms—for example, chloro, bromo or iodo. Suitably the halogen atoms will be at the ends of the chain.

By “solvent-free”, we mean that no solvent is specifically added to perform the reaction step. Thus, the reaction is free of solvents such as acetone or acetonitrile as noted under Scheme 3 above. The reaction step is also free of solvents such as toluene, dichloromethane (DCM), dichloroethane (DCE), dimethylformamide (DMF), methanol, acetic acid, methanol/aqueous acidic systems such as those comprising methanol and acetic acid; or mixtures of any two or more of the above solvents. The reactants themselves i.e. the cyclic amine and haloalkane compounds, and any additional compounds, such as an organic base as discussed below, are not encompassed by the term “solvent”.

The cyclic amine compound preferably comprises a compound of formula I or salts thereof:

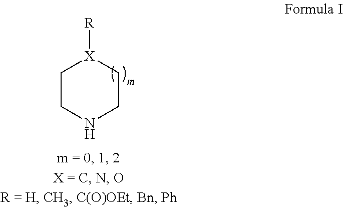

where Et=ethyl; Bn=benzyl and Ph=phenyl. Some specific example of suitable compounds are given below.

The process is preferably carried out in a single reaction step—that is, there is no need for two sequential chemical reactions when forming a compound of formula II from a compound of formula I.

In one aspect, the process further comprises the presence of an organic base. Suitable organic bases are given below.

Thus, according to a further aspect of the present invention, there is provided a process for the preparation of a compound of formula II:

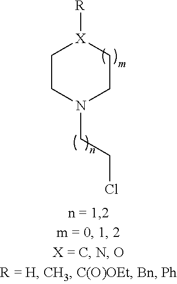

where Et=ethyl; Bn=benzyl and Ph=phenyl comprising the step of reacting a cyclic amine of formula I or salt thereof with an

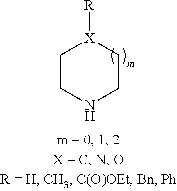

(where Et=ethyl; Bn=benzyl and Ph=phenyl) alkylating agent to form a compound of formula II, wherein the process is carried out in the presence of an organic base and is solvent-free.

The process of the invention may be carried out in batch mode or carried out in continuous mode, for example as a continuous flow procedure. Any suitable continuous flow apparatus may be used, for example a continuous flow reactor, and these and their operation are well known.

According to another aspect of the present invention, there are processes provided comprising
a) reacting cyclic amines with 1-bromo-2-chloroethane or 1-bromo-3-chloropropane in the presence of an organic base under solvent-free, batch conditions to form 1-chloroethyl or 1-chloropropyl substituted cyclic amines, or
b) reacting cyclic amines with 1-bromo-2-chloroethane or 1-bromo-3-chloropropane in the presence of an organic base under solvent-free, continuous flow conditions to form 1-chloroethyl or 1-chloropropyl substituted cyclic amines.

Surprisingly, it has been found that the present invention affords 1-chloroalkyl substituted cyclic amines in higher yields and with lower amount of dimeric side product (such as IIIa) than the processes disclosed previously without additional process steps (such as protection and deprotection, reduction etc.), without needing to use extreme temperatures and undesirable reagents (such as corrosive reagents, toxic reagents or methanol/aqueous acidic systems).

In one aspect, for example option b) above, of the present invention the process is carried out in continuous flow mode, thus providing flexibility for the method of production. Surprisingly, the invention enables a solution for the technical limitations (such as clogging due to the precipitation of the salt formed from the base) of continuous flow processes by the selection of organic base and solvent-free conditions. As novel features, in continuous flow mode the impurity content is significantly decreased, the reaction time is extremely reduced compared to the processes disclosed previously, and the productivity is thereby highly improved.

The present invention controls the formation of undesirable dimeric side products (such as IIIa). 1-Chloroalkyl substituted cyclic amines obtained by the process of the present invention (whether in batch or continuous mode) can, for example, be either purified (eg. by column chromatography) or used directly without purification.

Consequently, in either batch or continuous mode, in the present invention the reaction time is reduced, the use of solvent is avoided and the concentration of the starting material is increased due to solvent-free conditions. Therefore the purity, the chemical yield and the productivity are highly improved and consistent, the waste is reduced and the excess of the alkylating agent, such as 1-bromo-2-chloroethane or 1-bromo-3-chloropropane, may be optionally recycled and reused.

### DETAILED DESCRIPTION OF THE INVENTION

The present invention provides alternative processes for preparing 1-chloroalkyl substituted cyclic amines, particularly those of formula II.

More specifically, in preferred aspects but not limited to these examples, the present invention may provide processes comprising:
a) reacting 4-alkylpiperidine with 1-bromo-2-chloroethane in the presence of an organic base to form 1-(2-chloroethyl)-4-alkylpiperidine; or
b) reacting 4-alkylpiperidine with 1-bromo-3-chloropropane in the presence of an organic base to form 1-(3-chloropropyl)-4-alkylpiperidine; or
c) reacting alkyl 4-piperidinecarboxylate with 1-bromo-2-chloroethane in the presence of an organic base to form alkyl 1-(2-chloroethyl) piperidine-4-carboxylate; or
d) reacting alkyl 4-piperidinecarboxylate with 1-bromo-3-chloropropane in the presence of an organic base to form alkyl 1-(3-chloropropyl) piperidine-4-carboxylate; or
e) reacting 1-alkylpiperazine with 1-bromo-2-chloroethane in the presence of an organic base to form 1-(2-chloroethyl)-4-alkylpiperazine; or
f) reacting 1-alkylpiperazine with 1-bromo-2-chloropropane in the presence of an organic base to form 1-(3-chloropropyl)-4-alkylpiperazine; or
g) reacting 1-arylpiperazine with 1-bromo-2-chloroethane in the presence of an organic base to form 1-(2-chloroethyl)-4-arylpiperazine; or
h) reacting 1-arylpiperazine with 1-bromo-3-chloropropane in the presence of an organic base to form 1-(3-chloropropyl)-4-arylpiperazine; or
i) reacting morpholine with 1-bromo-2-chloroethane in the presence of an organic base to form 1-(2-chloroethyl)morpholine; or
j) reacting morpholine with 1-bromo-3-chloropropane in the presence of an organic base to form 1-(3-chloropropyl)morpholine; or
k) reacting pyrrolidine with 1-bromo-2-chloroethane in the presence of an organic base to form 1-(2-chloroethyl)pyrrolidine; or
l) reacting pyrrolidine with 1-bromo-3-chloropropane in the presence of an organic base to form 1-(3-chloropropyl)pyrrolidine; or
m) reacting hexamethyleneimine with 1-bromo-2-chloroethane in the presence of an organic base to form 1-(2-chloroethyl)hexamethyleneimine; or
n) reacting hexamethyleneimine with 1-bromo-3-chloropropane in the presence of an organic base to form 1-(3-chloropropyl)hexamethyleneimine.

In another aspect of the invention, a process for the preparation of 1-chloroalkyl substituted cyclic amines preferably comprises one of the following procedures, which are alternatives and may suitably be carried out in batch mode:
a) reacting 4-alkylpiperidine with 1-bromo-2-chloroethane in the presence of an organic base, at a temperature between about 20° C. and about 100° C., to form 1-(2-chloroethyl)-4-alkylpiperidine; and thereafter removing salts so formed from reaction mixture, preferably by performing filtration and/or aqueous extraction, and isolating 1-(2-chloroethyl)-4-alkylpiperidine from the resulting solution, preferably by concentration; or
b) reacting 4-alkylpiperidine with 1-bromo-3-chloropropane in the presence of an organic base, at a temperature between about 20° C. and about 100° C., to form 1-(3-chloropropyl)-4-alkylpiperidine; and thereafter removing salts so formed from reaction mixture, preferably by performing filtration and/or aqueous extraction, and isolating 1-(3-chloropropyl)-4-alkylpiperidine from the resulting solution, preferably by concentration; or
c) reacting alkyl 4-piperidinecarboxylate with 1-bromo-2-chloroethane in the presence of an organic base, at a temperature between about 20° C. and about 100° C., to form alkyl 1-(2-chloroethyl)piperidine-4-carboxylate; and thereafter removing salts so formed from reaction mixture, preferably by performing filtration and/or aqueous extraction, and isolating alkyl 1-(2-chloroethyl)piperidine-4-carboxylate from the resulting solution, preferably by concentration; or
d) reacting alkyl 4-piperidinecarboxylate with 1-bromo-3-chloropropane in the presence of an organic base, at a temperature between about 20° C. and about 100° C., to form alkyl 1-(3-chloropropyl)piperidine-4-carboxylate; and thereafter removing salts so formed from reaction mixture, preferably by performing filtration and/or aqueous extraction, and isolating alkyl 1-(3-chloropropyl)piperidine-4-carboxylate from the resulting solution, preferably by concentration; or
e) reacting 1-alkylpiperazine with 1-bromo-2-chloroethane in the presence of an organic base, at a temperature between about 20° C. and about 100° C., to form 1-(2-chloroethyl)-4-alkylpiperazine; and thereafter removing salts so formed from reaction mixture, preferably by performing filtration and/or aqueous extraction, and isolating 1-(2-chloroethyl)-4-alkylpiperazine from the resulting solution, preferably by concentration; or
f) reacting 1-alkylpiperazine with 1-bromo-3-chloropropane in the presence of an organic base, at a temperature between about 20° C. and about 100° C., to form 1-(3-chloropropyl)-4-alkylpiperazine; and thereafter removing salts so formed from reaction mixture, preferably by performing filtration and/or aqueous extraction, and isolating 1-(3-chloropropyl)-4-alkylpiperazine from the resulting solution, preferably by concentration; or
g) reacting 1-arylpiperazine with 1-bromo-2-chloroethane in the presence of an organic base, at a temperature between about 20° C. and about 100° C., to form 1-(2-chloroethyl)-4-arylpiperazine; and thereafter removing salts so formed from reaction mixture, preferably by performing filtration and/or aqueous extraction, and isolating 1-(2-chloroethyl)-4-arylpiperazine from the resulting solution, preferably by concentration; or
h) reacting 1-arylpiperazine with 1-bromo-3-chloropropane in the presence of an organic base, at a temperature between about 20° C. and about 100° C., to form 1-(3-chloropropyl)-4-arylpiperazine; and thereafter removing salts so formed from reaction mixture, preferably by performing filtration and/or aqueous extraction, and isolating 1-(3-chloropropyl)-4-arylpiperazine from the resulting solution, preferably by concentration; or
i) reacting morpholine with 1-bromo-2-chloroethane in the presence of an organic base, at a temperature between about 20° C. and about 100° C., to form 1-(2-chloroethyl)morpholine; and thereafter removing salts so formed from reaction mixture, preferably by performing filtration and/or aqueous extraction, and isolating 1-(2-chloroethyl)morpholine from the resulting solution, preferably by concentration; or
j) reacting morpholine with 1-bromo-3-chloropropane in the presence of an organic base, at a temperature between about 20° C. and about 100° C., to form 1-(3-chloropropyl)morpholine; and thereafter removing salts so formed from reaction mixture, preferably by performing filtration and/or aqueous extraction, and isolating 1-(3-chloropropyl)morpholine from the resulting solution, preferably by concentration; or
k) reacting pyrrolidine with 1-bromo-2-chloroethane in the presence of an organic base, at a temperature between about 20° C. and about 100° C., to form 1-(2-chloroethyl)pyrrolidine; and thereafter removing salts so formed from reaction mixture, preferably by performing filtration and/or aqueous extraction, and isolating 1-(2-chloroethyl)pyrrolidine from the resulting solution, preferably by concentration; or
l) reacting pyrrolidine with 1-bromo-3-chloropropane in the presence of an organic base, at a temperature between about 20° C. and about 100° C., to form 1-(3-chloropropyl)pyrrolidine; and thereafter removing salts so formed from reaction mixture, preferably by performing filtration and/or aqueous extraction, and isolating 1-(3-chloropropyl)pyrrolidine from the resulting solution, preferably by concentration; or
m) reacting hexamethyleneimine with 1-bromo-2-chloroethane in the presence of an organic base, at a temperature between about 20° C. and about 100° C., to form 1-(2-chloroethyl)hexamethyleneimine; and thereafter removing salts so formed from reaction mixture, preferably by performing filtration and/or aqueous extraction, and isolating 1-(2-chloroethyl)hexamethyleneimine from the resulting solution, preferably by concentration; or
n) reacting hexamethyleneimine with 1-bromo-3-chloropropane in the presence of an organic base, at a temperature between about 20° C. and about 100° C., to form 1-(3-chloropropyl)hexamethyleneimine; and thereafter removing salts so formed from reaction mixture, preferably by performing filtration and/or aqueous extraction, and isolating 1-(3-chloropropyl)hexamethyleneimine from the resulting solution, preferably by concentration.

Any suitable organic base may be used for the processes and reactions described above. The organic base used in batch mode for examples (a)-(n) may, for example, be selected from the group consisting of organic bases such as amines like N,N-diisopropylethylamine, triethylamine, tributylamine, N-methylimidazole, 4-(dimethylamino)pyridine, 1,8-diazabicyclo[5.4.0]undec-7-ene, 1,5-diazabicyclo[4.3.0]non-5-ene. Preferably the organic base is N,N-diisopropylethylamine or triethylamine. Upon completion of the reaction, N,N-diisopropylethylamine or triethylamine salts can be removed, preferentially by filtration and aqueous extraction, and the resulting solution is concentrated to isolate the 1-chloroalkyl substituted cyclic amine.

In the process of the invention, preferably an excess of alkylating agent is used, in relation to the cyclic amine. Preferably, an excess of 5 or more, by molar equivalent, is used. An excess of 8 or 9 or 10 or more may be used. For example, by using excess of the bifunctional alkylating agent between about 5 to about 15 equivalents, in the above examples it is possible to obtain the respective products in yields between 33 and 94% with a residual content of dimeric side product between 0 and 23%.

Additionally, the excess of the alkylating agent, such as 1-bromo-2-chloroethane or 1-bromo-3-chloropropane, may be optionally recycled and reused.

In another aspect of the invention, a process for the preparation of 1-chloroalkyl substituted cyclic amines preferably comprises one of the following procedures, which are alternatives and may suitably be carried out in continuous flow mode:
a) reacting 1-bromo-2-chloroethane with 4-alkylpiperidine mixed with an organic base in a continuous flow reactor, at a temperature between about 60° C. and about 100° C., to form 1-(2-chloroethyl)-4-alkylpiperidine; and thereafter removing salts so formed from reaction mixture, preferably by performing filtration and/or aqueous extraction, and isolating 1-(2-chloroethyl)-4-alkylpiperidine from the resulting solution, preferably by concentration; or
b) reacting 1-bromo-3-chloropropane with 4-alkylpiperidine mixed with an organic base in a continuous flow reactor, at a temperature between about 60° C. and about 100° C., to form 1-(3-chloropropyl)-4-alkylpiperidine; and thereafter removing salts so formed from reaction mixture, preferably by performing filtration and/or aqueous extraction, and isolating 1-(3-chloropropyl)-4-alkylpiperidine from the resulting solution, preferably by concentration; or
c) reacting 1-bromo-2-chloroethane with alkyl 4-piperidinecarboxylate mixed with an organic base in a continuous flow reactor, at a temperature between about 60° C. and about 100° C., to form alkyl 1-(2-chloroethyl)piperidine-4-carboxylate; and thereafter removing salts so formed from reaction mixture, preferably by performing filtration and/or aqueous extraction, and isolating alkyl 1-(2-chloroethyl)piperidine-4-carboxylate from the resulting solution, preferably by concentration; or
d) reacting 1-bromo-3-chloropropane with alkyl 4-piperidinecarboxylate mixed with an organic base in a continuous flow reactor, at a temperature between about 60° C. and about 100° C., to form alkyl 1-(3-chloropropyl)piperidine-4-carboxylate; and thereafter removing salts so formed from reaction mixture, preferably by performing filtration and/or aqueous extraction, and isolating alkyl 1-(3-chloropropyl)piperidine-4-carboxylate from the resulting solution, preferably by concentration; or
e) reacting 1-bromo-2-chloroethane with 1-alkylpiperazine mixed with an organic base in a continuous flow reactor, at a temperature between about 60° C. and about 100° C., to form 1-(2-chloroethyl)-4-alkylpiperazine; and thereafter removing salts so formed from reaction mixture, preferably by performing filtration and/or aqueous extraction, and isolating 1-(2-chloroethyl)-4-alkylpiperazine from the resulting solution, preferably by concentration; or
f) reacting 1-bromo-3-chloropropane with 1-alkylpiperazine mixed with an organic base in a continuous flow reactor, at a temperature between about 60° C. and about 100° C., to form 1-(3-chloropropyl)-4-alkylpiperazine; and thereafter removing salts so formed from reaction mixture, preferably by performing filtration and/or aqueous extraction, and isolating 1-(3-chloropropyl)-4-alkylpiperazine from the resulting solution, preferably by concentration; or
g) reacting 1-bromo-2-chloroethane with 1-arylpiperazine mixed with an organic base in a continuous flow reactor, at a temperature between about 60° C. and about 100° C., to form 1-(2-chloroethyl)-4-arylpiperazine; and thereafter removing salts so formed from reaction mixture, preferably by performing filtration and/or aqueous extraction, and isolating 1-(2-chloroethyl)-4-arylpiperazine from the resulting solution, preferably by concentration; or
h) reacting 1-bromo-3-chloropropane with 1-arylpiperazine mixed with an organic base in a continuous flow reactor, at a temperature between about 60° C. and about 100° C., to form 1-(3-chloropropyl)-4-arylpiperazine; and thereafter removing salts so formed from reaction mixture, preferably by performing filtration and/or aqueous extraction, and isolating 1-(3-chloropropyl)-4-arylpiperazine from the resulting solution, preferably by concentration; or
i) reacting 1-bromo-2-chloroethane with morpholine mixed with an organic base in a continuous flow reactor, at a temperature between about 60° C. and about 100° C., to form 1-(2-chloroethyl)morpholine; and thereafter removing salts so formed from reaction mixture, preferably by performing filtration and/or aqueous extraction, and isolating 1-(2-chloroethyl)morpholine from the resulting solution, preferably by concentration; or
j) reacting 1-bromo-3-chloropropane with morpholine mixed with an organic base in a continuous flow reactor, at a temperature between about 60° C. and about 100° C., to form 1-(3-chloropropyl)morpholine; and thereafter removing salts so formed from reaction mixture, preferably by performing filtration and/or aqueous extraction, and isolating 1-(3-chloropropyl)morpholine from the resulting solution, preferably by concentration; or
k) reacting 1-bromo-2-chloroethane with pyrrolidine mixed with an organic base in a continuous flow reactor, at a temperature between about 60° C. and about 100° C., to form 1-(2-chloroethyl)pyrrolidine; and thereafter removing salts so formed from reaction mixture, preferably by performing filtration and/or aqueous extraction, and isolating 1-(2-chloroethyl)pyrrolidine from the resulting solution, preferably by concentration; or
l) reacting 1-bromo-3-chloropropane with pyrrolidine mixed with an organic base in a continuous flow reactor, at a temperature between about 60° C. and about 100° C., to form 1-(3-chloropropyl)pyrrolidine; and thereafter removing salts so formed from reaction mixture, preferably by performing filtration and/or aqueous extraction, and isolating 1-(3-chloropropyl)pyrrolidine from the resulting solution, preferably by concentration; or
m) reacting 1-bromo-2-chloroethane with hexamethyleneimine mixed with an organic base in a continuous flow reactor, at a temperature between about 60° C. and about 100° C., to form 1-(2-chloroethyl)hexamethyleneimine; and thereafter removing salts so formed from reaction mixture, preferably by performing filtration and/or aqueous extraction, and isolating 1-(2-chloroethyl)hexamethyleneimine from the resulting solution, preferably by concentration; or
n) reacting 1-bromo-3-chloropropane with hexamethyleneimine mixed with an organic base in a continuous flow reactor, at a temperature between about 60° C. and about 100° C., to form 1-(3-chloropropyl)hexamethyleneimine; and thereafter removing salts so formed from reaction mixture, preferably by performing filtration and/or aqueous extraction, and isolating 1-(3-chloropropyl)hexamethyleneimine from the resulting solution, preferably by concentration.

Any suitable organic base may be used for the processes and reactions described above. The organic base used in continuous flow mode for step (a)-(n) may, for example, be selected from the group consisting of organic bases such as amines like N,N-diisopropylethylamine, triethylamine, tributylamine, N-methylimidazole, 4-(dimethylamino) pyridine, 1,8-diazabicyclo[5.4.0]undec-7-ene, 1,5-diazabicyclo[4.3.0]non-5-ene. Preferably the organic base is 1,8-diazabicyclo[5.4.0]undec-7-ene or 1,5-diazabicyclo[4.3.0]non-5-ene. 1,8-Diazabicyclo[5.4.0]undec-7-ene or 1,5-diazabicyclo[4.3.0]non-5-ene salts can be removed by an aqueous quench and a following liquid-liquid extraction. The resulting organic phase is concentrated to isolate the 1-chloroalkyl substituted cyclic amine.

In the process of the invention, preferably an excess of alkylating agent is used, in relation to the cyclic amine. Preferably, an excess of 5 or more, by molar equivalent, is used. An excess of 8 or 9 or 10 or more may be used. For example, by using excess of the bifunctional alkylating agent between about 5 to about 15 equivalents, in the above examples it is possible to obtain the respective products in yields between 55 and 80% with a residual content of dimeric side product between 3 and 5%.

It will be understood that in a continuous flow reactor, the flow rate has to be adjusted in order to obtain an optimal residence time of the reaction mixture in the continuous flow reactor with the aim of completing the reaction. Flow and pressure ranges used are characteristics of the reaction model. For example, in the case of a custom-made PFA coil reactor, typically the flow is in the range of 0.1 to 1.00 mL/min and the pressure is in the range from 1 to 4 bar.

Additionally, the excess of the alkylating agent, such as 1-bromo-2-chloroethane or 1-bromo-3-chloropropane, may be optionally recycled and reused.

The mode of operation herein disclosed comprises the use of a large excess of the (for example) di-halo alkylating agent, for example about 9 or 10 molar equivalents or more with respect to the substrate, to minimize the formation of the dimer impurity, for both continuous flow chemistry procedures and batch mode procedures. Under the conditions of this mode of operation, to obtain the desired product with the lowest content of dimer, as will be understood, minor adjustments to the reaction temperature, residence time and reagents stream can be made in order to optimize the process for a given specific reaction. Dimer content as measured by GC is suitably equal to, or lower than 23%, preferably, equal to, or lower than 9% and more preferably, equal to, or lower than 5%.

The mode of operation of the present invention also comprises the use of an appropriate organic amine acting as a base, to address the technical problem of clogging, occasionally observed in small scale flow chemistry procedures. Preferably, the appropriate amine may be any one of the amines from the group consisting of N,N-diisopropylethylamine, triethylamine, tributylamine, N-methylimidazole, 4-(dimethylamino)pyridine, 1,8-diazabicyclo[5.4.0]undec-7-ene, 1,5-diazabicyclo[4.3.0]non-5-ene. We have found that the use of any of these amines prevents tubes obstruction in flow chemistry mode.

A quantum mechanics study was also performed with the software Gaussian 16¹ at the B3LYP/3-21G level of theory to develop a scientific rational to help support our experimental findings, and we have looked at the synthesis of haloethyl derivatives of piperidine, pyrrolidine and azepane from this perspective. The application of Quantum Mechanics theory to the study of the mechanisms involved in chemical reactions enables predicting the outcome of chemical reactions through the calculation of energy barriers and the calculation of overall energy balances. The energy barrier provides a quantitative relationship for reactions kinetics and the overall energy balance provides a quantitative relationship for the reactions' thermodynamics. The energy barrier is calculated by the difference between the transition state energy and the reactants energy minimum. The reactants energy minimum corresponds to the most stable geometric configuration when the reactants approach each other. The overall energy balance is calculated by the difference between the products energy and the reactants energy. If the overall energy balance is a positive, the reaction is designated as endergonic and it means that it is necessary to supply energy to the system for the reaction to occur. In other words, the reaction is not spontaneous, at the temperature selected for the quantum mechanics calculations, and the reactants are more stable than the products. If the overall energy balance is negative, the reaction is designated as exergonic and that means that it is not necessary to supply energy for the reaction to occur. In other words, the conversion of the reactants into the products is spontaneous, at the temperature selected for the quantum mechanics calculations, and the products are more stable than the reactants. If the quantum mechanics study is also applied to the side-reactions, reliable quantitative co-relations can be established between the main reaction and the side reactions. Such data enable prediction of the main reaction selectivity and the formation of impurities. ¹Gaussian 16, Revision C.01, M. J. Frisch, G. W. Trucks, H. B. Schlegel, G. E. Scuseria, M. A. Robb, J. R. Cheeseman, G. Scalmani, V. Barone, G. A. Petersson, H. Nakatsuji, X. Li, M. Caricato, A. V. Marenich, J. Bloino, B. G. Janesko, R. Gomperts, B. Mennucci, H. P. Hratchian, J. V. Ortiz, A. F. Izmaylov, J. L. Sonnenberg, D. Williams-Young, F. Ding, F. Lipparini, F. Egidi, J. Goings, B. Peng, A. Petrone, T. Henderson, D. Ranasinghe, V. G. Zakrzewski, J. Gao, N. Rega, G. Zheng, W. Liang, M. Hada, M. Ehara, K. Toyota, R. Fukuda, J. Hasegawa, M. Ishida, T. Nakajima, Y. Honda, O. Kitao, H. Nakai, T. Vreven, K. Throssell, J. A. Montgomery, Jr., J. E. Peralta, F. Ogliaro, M. J. Bearpark, J. J. Heyd, E. N. Brothers, K. N. Kudin, V. N. Staroverov, T. A. Keith, R. Kobayashi, J. Normand, K. Raghavachari, A. P. Rendell, J. C. Burant, S. S. Iyengar, J. Tomasi, M. Cossi, J. M. Millam, M. Klene, C. Adamo, R. Cammi, J. W. Ochterski, R. L. Martin, K. Morokuma, O. Farkas, J. B. Foresman, and D. J. Fox, Gaussian, Inc., Wallingford CT, 2016.

To evaluate the selectivity of the main reaction, quantum mechanics calculations were performed on the synthesis of some halo ethyl derivatives of different cyclic amines. Specifically, calculations were performed on the reaction of ethyl isonipecotate with 1bromo-2-chloroethane, which was tested in the laboratory (see, for instance, Examples 1 and 10), as well as on the reactions of pyrrolidine, piperidine and azepane with 1-bromo-2-chloroethane, respectively. The reaction tested in the laboratory is considered as the reference case. The quantum mechanics calculations were also done on the reactions of ethyl isonipecotate, with 1-bromo-2-iodoethane, 1,2-dibromoethane, 1,2-diiodoethane and 1-bromo-2-fluoroethane, respectively. The quantum mechanics calculations addressed the main reaction (attack of the nitrogen cyclic amine on the di-halo ethyl carbon attached to the better halogen leaving group), the secondary reaction (attack of the nitrogen cyclic amine on the di-halo ethyl carbon attached to the worst halogen leaving group) and the side-reactions leading to the formation of the dimer impurity, namely the reactions of the cyclic amine starting material with either the main halo ethyl product and the secondary halo ethyl product. The scheme below depicts the transformations referred to above for the reaction between ethyl isonipecotate and 1-bromo-2-chloroethane.

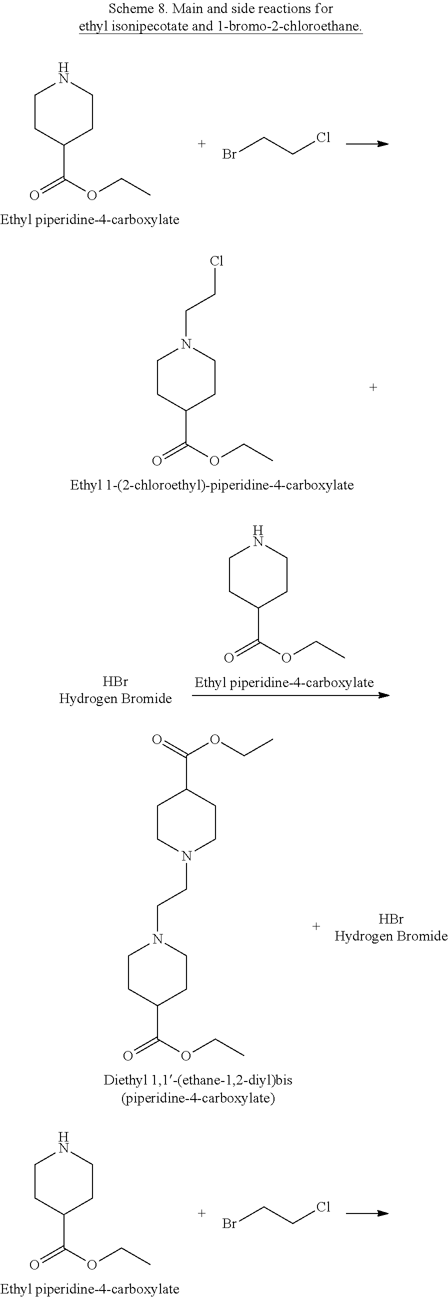

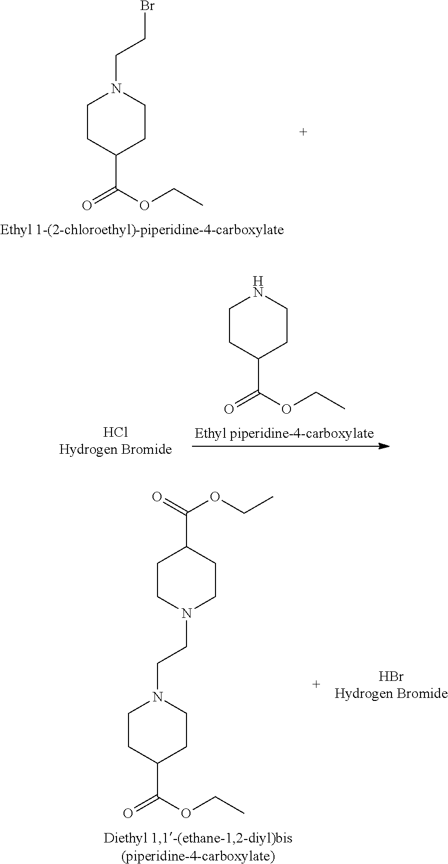 [SMILES: CCOC(=O)C1CCN(CCBr)CC1 ; CCOC(=O)C1CCNCC1 ; CCOC(=O)C1CCN(CCN2CCC(C(=O)OCC)CC2)CC1]

The quantum mechanics calculations were performed at the B3LYP/3-21G level of theory and at the default temperature (298.15 K; 25° C.). The energy values used for the calculations were those generated by the software with thermal corrections. The quantum mechanics study outputs together with the experimental results disclosed in the present application provide insight into the likelihood of success of the chemical reactions, and thus are also of predictive value.

The energy barrier calculated for the reaction between ethyl isonipecotate and 1-bromo-2-chloroethane was 80.5 kJ/mol; comparable to those calculated for the reactions of piperidine (82.5 kJ/mol) and pyrrolidine (80.4 kJ/mol), and higher than that calculated for azepane (68.1 kJ/mol), as presented in Table 1.

TABLE 1
Energy barriers and overall energy balances for 1-bromo-2-chloroethane main reaction.

| | | Overall |
|---|---|---|
| | Energy | Energy |
| | Barrier | Balance |
| Chemical Reaction Scheme | (kJ/mol) | kJ/mol) |
| 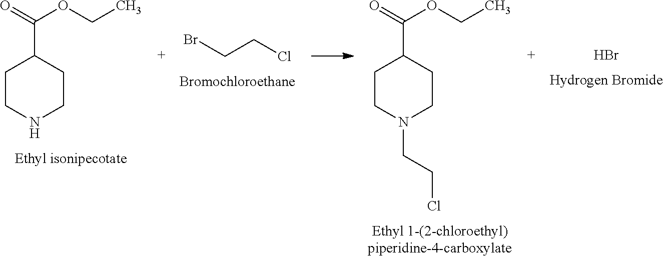 | 80.5 | 26.8 |
| 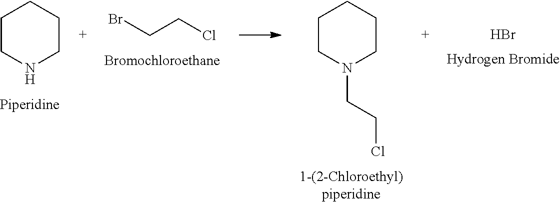 | 82.5 | −58.1 |
| 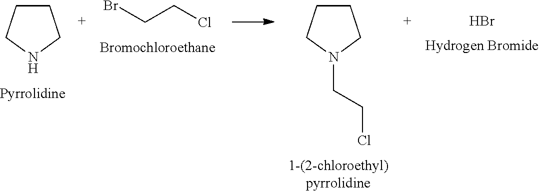 | 80.4 | 2.1 |
| 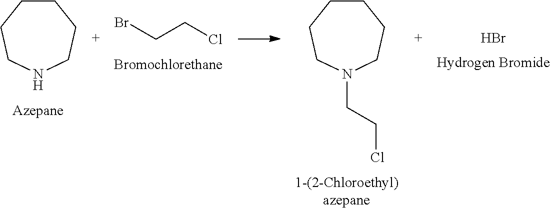 | 68.1 | 21.7 |

The energy barrier for the reaction tested experimentally is similar to those of the reactions of 1-bromo-2-chloroethane with the substrates piperidine and pyrrolidine, and it is higher than that calculated for azepane. The results show that the reaction between ethyl isonipecotate and 1-bromo-2-chloroethane is as much kinetically favorable as those of the reactions of piperidine and pyrrolidine with 1-bromo-2-chloroethane and it is slightly less energetically favorable than that of azepane with 1-bromo-2-chloromethane. In the case of azepane, the reaction with 1-bromo-2-chloroethane is more kinetically favorable than that of ethyl isonipecotate, and can be carried out at lower temperature. The Scheme 9 depicts the energy barriers and the overall energy balances for the abovementioned reactions with 1-bromo-2-chloroethane.

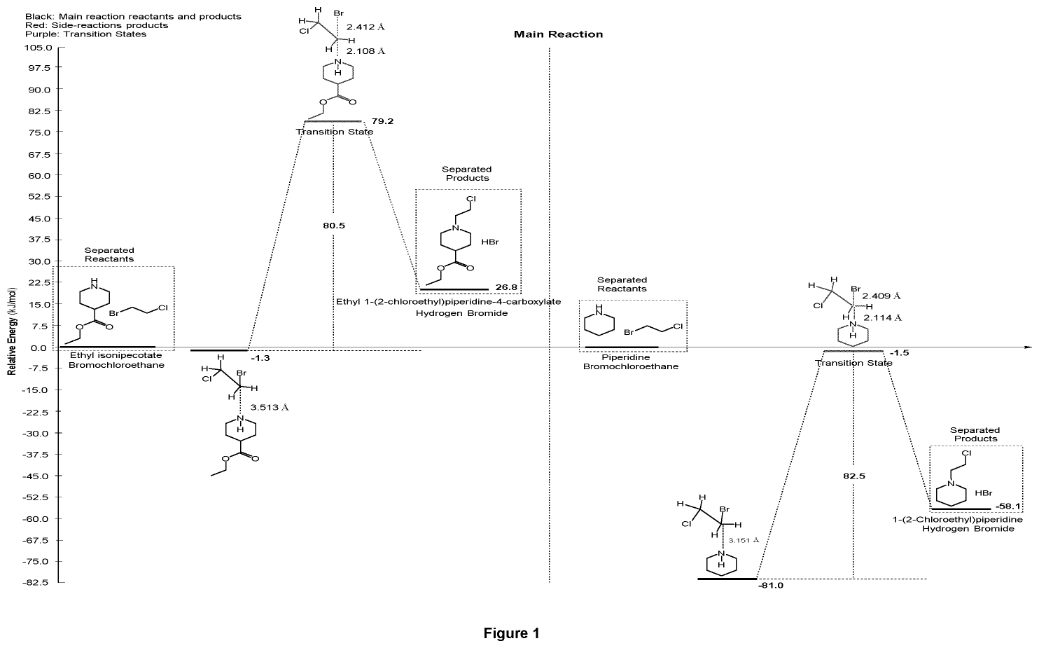

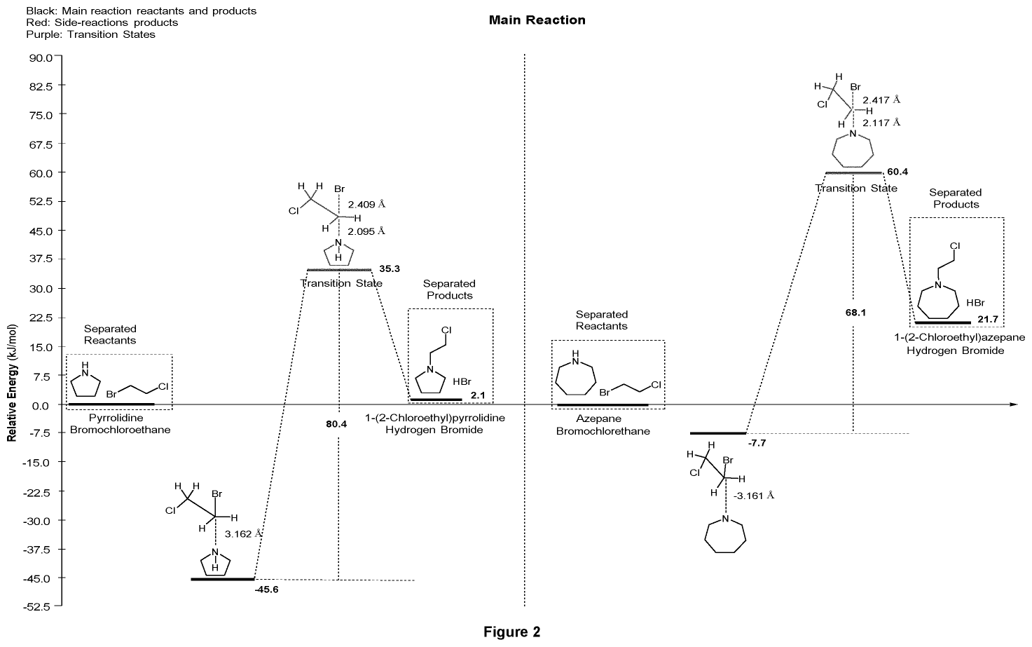

FIGS. 1 and 2 show: Energy graph for 1-bromo-2-chloroethane main reactions.

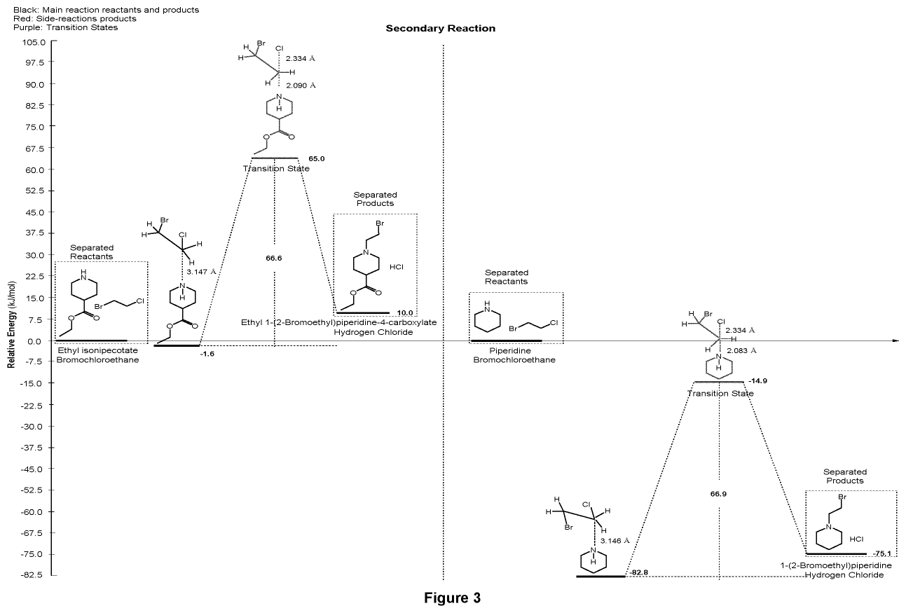

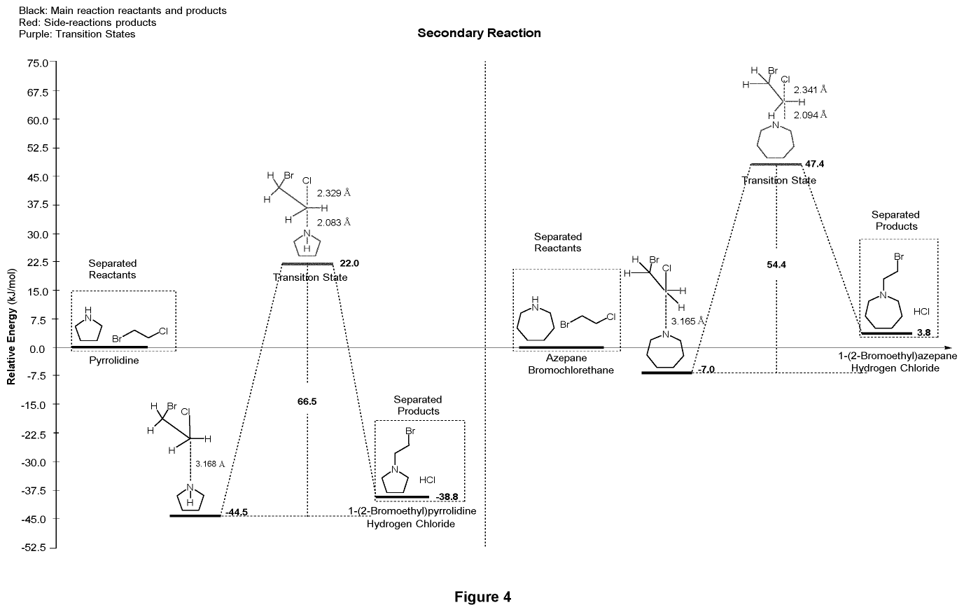

The energy barriers calculated for 1-bromo-2-chloroethane secondary reaction present a similar pattern to that observed for the main reaction: they are similar for ethyl isonipecotate, piperidine and pyrrolidine and more energetically favorable for azepane, as presented in Table 2 and FIGS. 3 and 4.

TABLE 2
Energy barriers and overall energy balances for 1-bromo-2-chloroethane secondary reaction.

| | Energy | Overall Energy |
|---|---|---|
| | Barrier | Balance |
| Chemical Reaction Scheme | (kJ/mol) | (kJ/mol) |
| 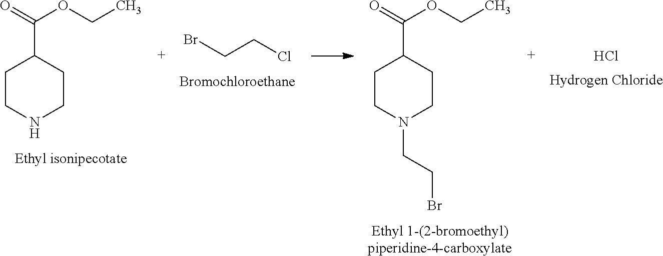 | 66.6 | 10.0 |
| 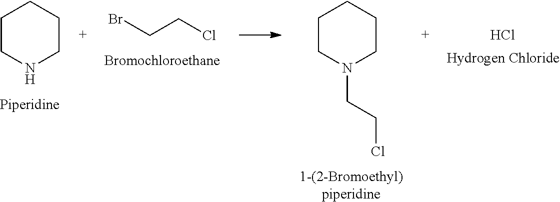 | 66.9 | −75.1 |
| 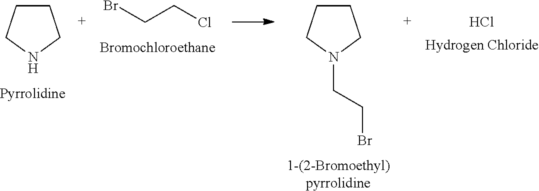 | 66.5 | −38.8 |
| 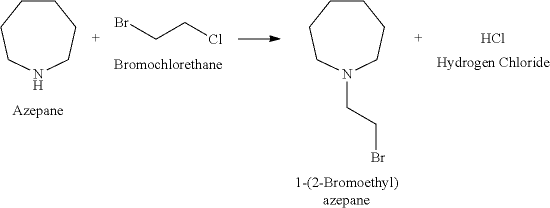 | 54.4 | 3.8 |

FIGS. 3 and 4 show: Energy graph for 1-bromo-2-chloroethane secondary reactions.

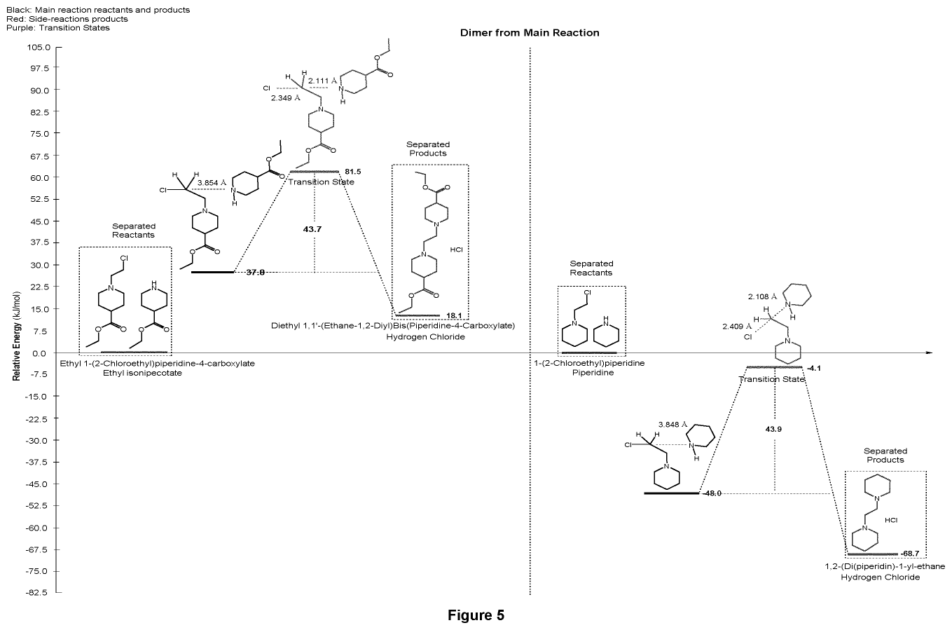

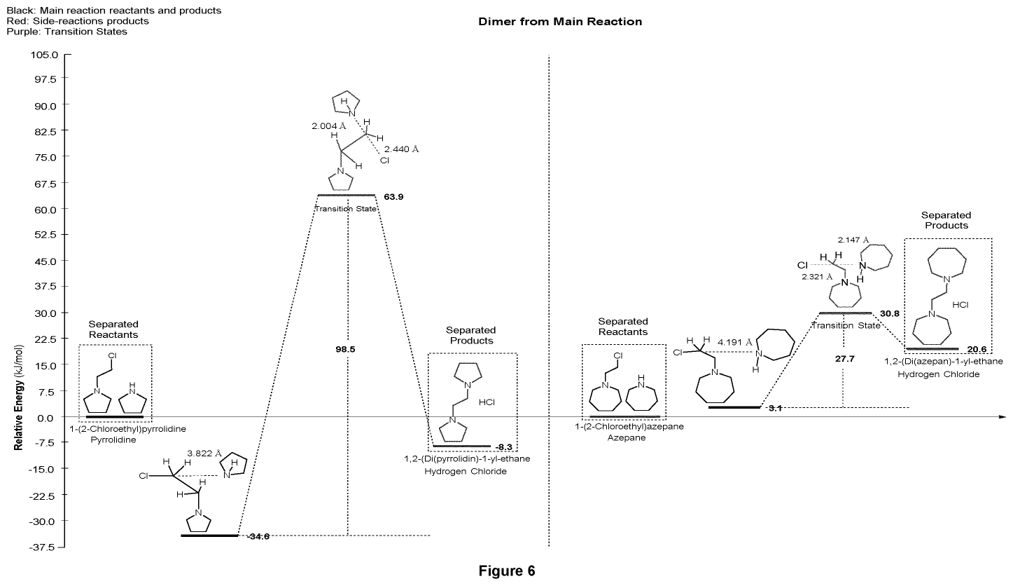

The energy barriers and the overall energy balances for the side reaction leading to the formation of the dimer impurity by reaction between the main product (chloroethyl derivative) with the cyclic amine starting material were calculated for the four cyclic amines, i.e., reaction between ethyl 1-(2-chloroethyl)piperidine-4-carboxylate and ethyl isonipecotate leading to the formation of the dimer diethyl 1,1′-(ethane-1,2-diyl)bis(piperidine-4-carboxylate), the reaction between 1-(2-chloroethyl)piperidine and piperidine leading to the formation of the dimer 1,2-Di(piperidin)-1-yl-ethane, the reaction between 1-(2-chloroethyl)pyrrolidine and pyrrolidine leading to the formation of the dimer 1,2-di(pyrrolidin)-1-yl-ethane and the reaction between 1,2-chloroethyl-azepane and azepane leading to the formation of the dimer 1,2-Di(azepane)-1-yl-ethane. The results are presented in the Table 3 and FIGS. 5 and 6.

TABLE 3
Energy barriers and overall energy balances for dimer formation via bromochloroethane main product.

| | | Overall |
|---|---|---|
| | Energy | Energy |
| | Barrier | Balance |
| Chemical Reaction Scheme | (kJ/mol) | (kJ/mol) |
| 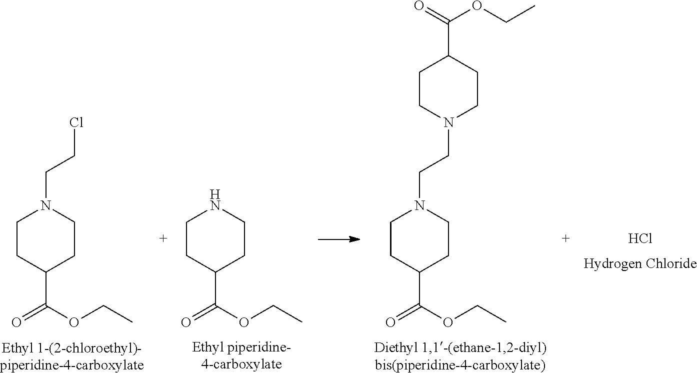 | 43.7 | 18.1 |
| 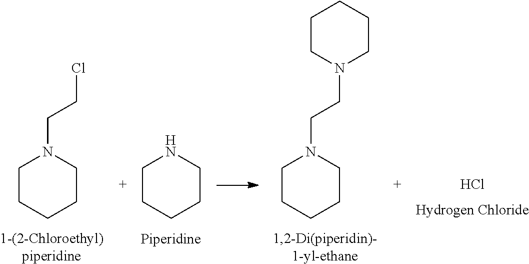 | 43.9 | −68.7 |
| 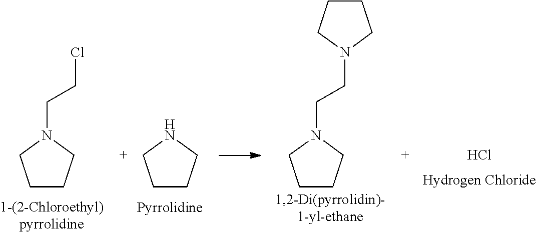 | 98.5 | −8.3 |
| 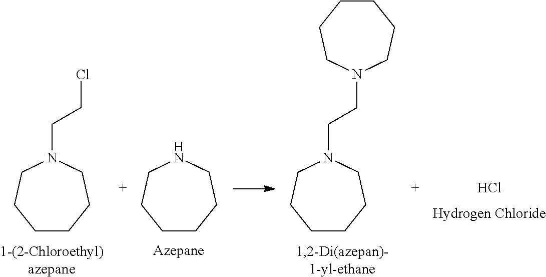 | 27.7 | 20.6 |

FIGS. 5 and 6 show: Energy graph for dimer formation via bromochloroethane main product.

The energy barrier for the formation of the dimer diethyl 1,1′-(ethane-1,2-diyl)bis(piperidine-4-carboxylate) is similar (43.7 kJ/mol) to that of the dimer 1,2-di(piperidin)-1-yl-ethane (43.9 kJ/mol). It is lower than the energy barrier for the formation of the dimer 1,2-di(pyrrolidin)-1-yl-ethane (98.5 kJ/mol), what means that the formation of the dimer 1,2-di(pyrrolidin)-1-yl-ethane is less favorable and its content in the product obtained from the main reaction is predicted to be lower than in the case of the dimer of the reference case. On the other hand, the formation of the dimer 1,2-(Diazepane)-1-yl-ethane is a little more energetically favorable what means that the content of this dimer in the main product 1-(2-chloroethyl)-azepane is expected to be a little higher than in the reference case. Nonetheless, adjustments in the process temperature and or residence time can be made to minimize the formation of the dimer. The experimental results obtained for the formation of the dimer in the reference case show that, under the conditions set forth in this application, the formation of the dimer was observed in levels between 0.3% and 8.5% (examples 1, 2, 3, 9, 10, 11, 12 and 13). Hence, process temperature can be slightly decreased and or the residence time can be slightly shortened for the synthesis of 1-(2-Chloroethyl)azepane to obtain levels of dimer within the range 0.3%-8.5%.

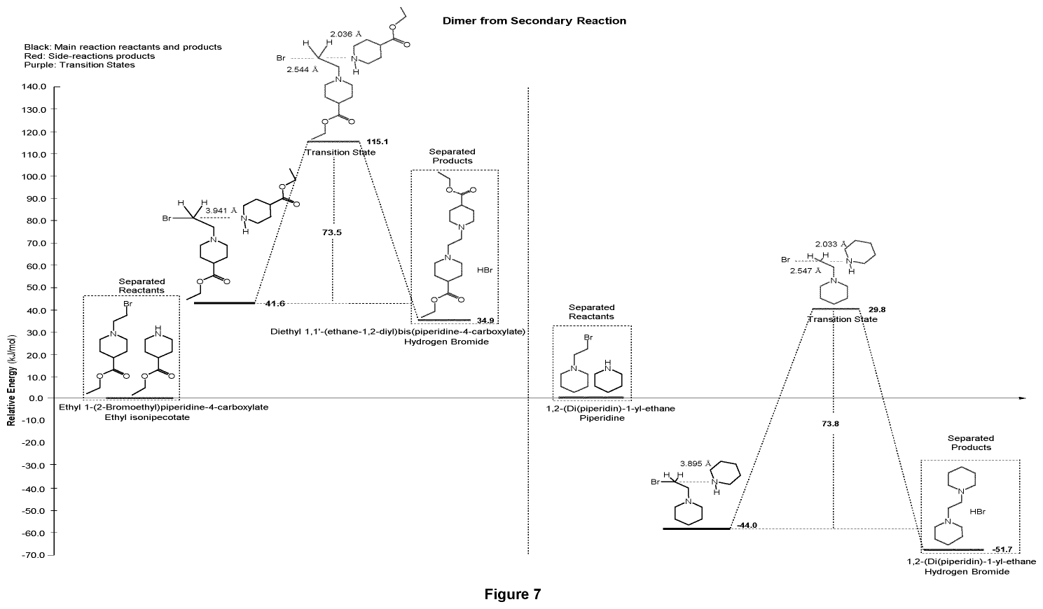

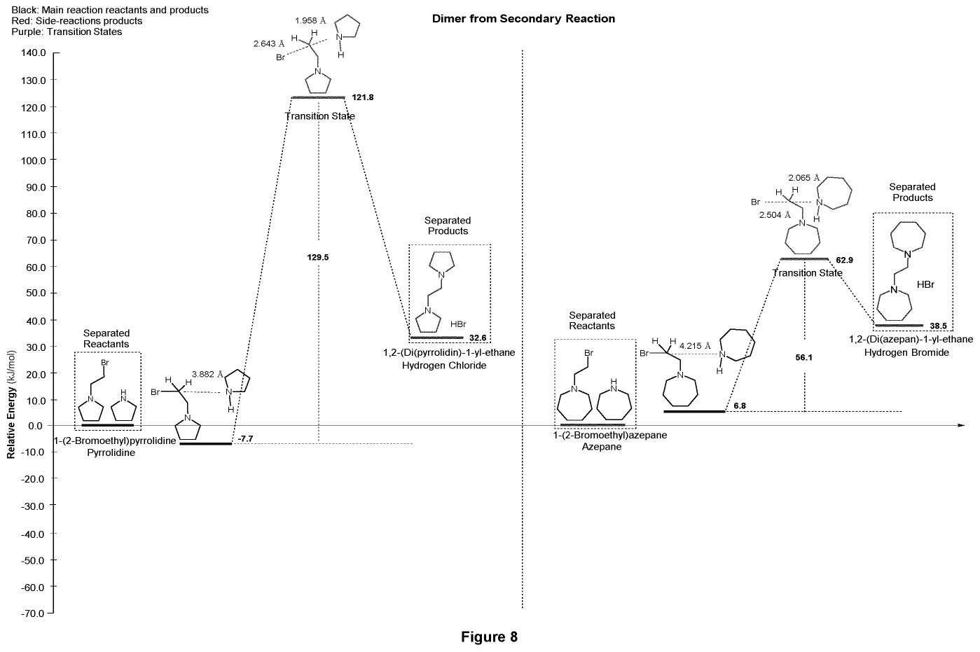

The energy barriers and the overall energy balances for the side reaction leading to the formation of the dimer impurity by reaction between the secondary product (bromoethyl derivative) with the cyclic amine starting material were calculated for the four cyclic amines, i.e., reaction between ethyl 1-(2-bromoethyl)piperidine-4-carboxylate and ethyl piperidine-4-carboxylate leading to the formation of the dimer diethyl 1,1′-(Ethane-1,2-Diyl)Bis(Piperidine-4-Carboxylate), the reaction between 1-(2-bromoethyl)piperidine and piperidine leading to the formation of the dimer 1,2-(Dipiperidin)-1-yl-ethane, the reaction between 1-(2-bromoethyl)pyrrolidine and pyrrolidine leading to the formation of the dimer 1,2-(Dipyrrolidin)-1-yl-ethane and the reaction between 1-(2-bromoethyl)-azepane and azepane leading to the formation of the dimer 1,2-Di(azepane)-1-yl-ethane. The results are presented in the Table 4 and FIGS. 7 and 8.

TABLE 4
Energy barriers and overall energy balances for dimer formation via 1-bromo-2-chloroethane secondary product.

| | | Overall |
|---|---|---|
| | Energy | Energy |
| | Barrier | Balance |
| Chemical Reaction Scheme | (kJ/mol) | (kJ/mol) |
| 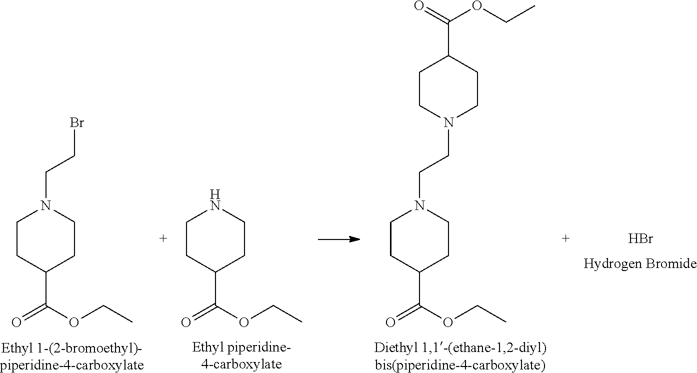 | 73.5 | 34.9 |
| 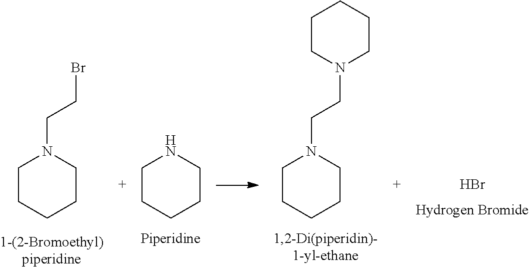 | 73.8 | −51.7 |
| 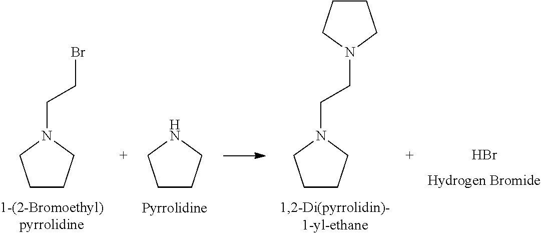 | 129.6 | 32.8 |
| 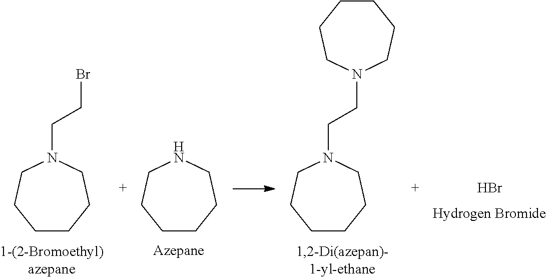 | 56.1 | 38.6 |

FIGS. 7 and 8 show: Energy graph for dimer formation via 1-bromo-2-chloroethane secondary product.

The energy barrier for the formation of the dimer diethyl 1,1′-ethane-1,2-diyl)bis(piperidine-4-carboxylate) is similar (73.5 kJ/mol) to that observed for the formation of the dimer 1,2-(dipiperidin)-1-yl-ethane (73.8 kJ/mol). It is lower than the energy barrier calculated for the formation of the dimer 1-2-(dipyrrolidin)-1-yl-ethane (129.6 kJ/mol), which means that the formation of this dimer is less favorable than the reference case. On the other hand, the formation of 1-2-(diazepan)-1-yl-ethane is a little more favorable (56.1 kJ/mol) than the reference case, predicting a little higher content of 1,2-(diazepan)-1-yl-ethane in the main product 1-(2-chloroethyl)-azepane. However, the process temperature can be slightly decreased and or the residence time can be slightly shorthened to minimize the formation of the dimer 1,2-(diazepan)-1-yl-ethane.

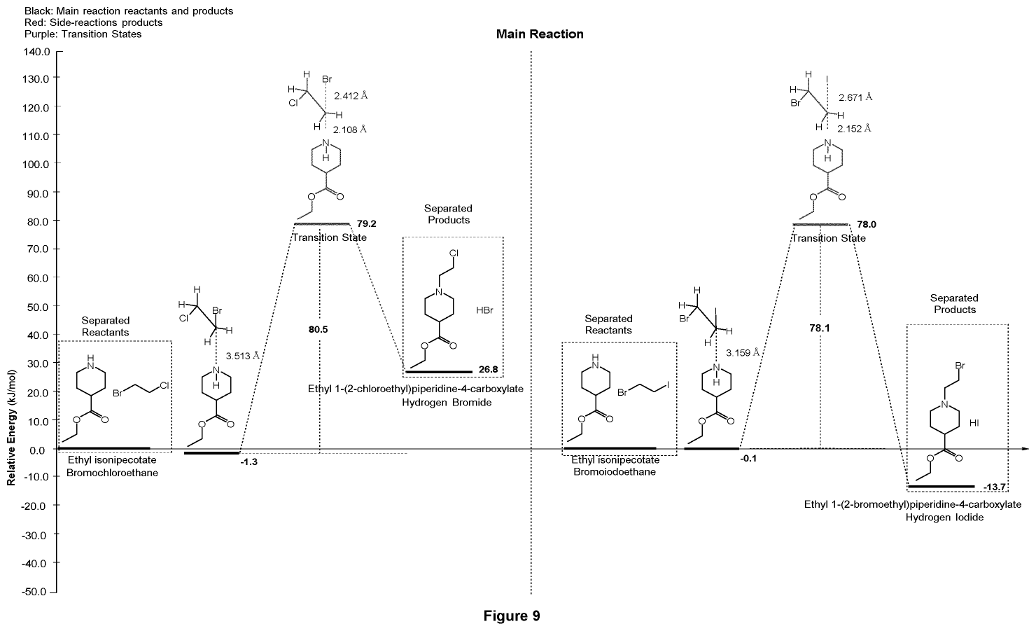

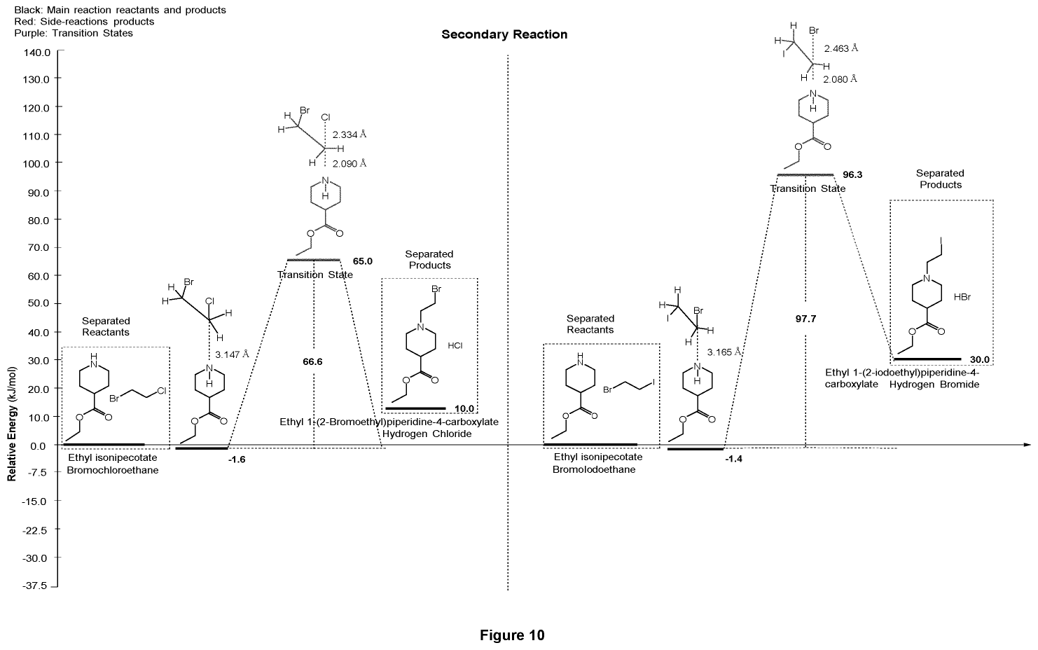

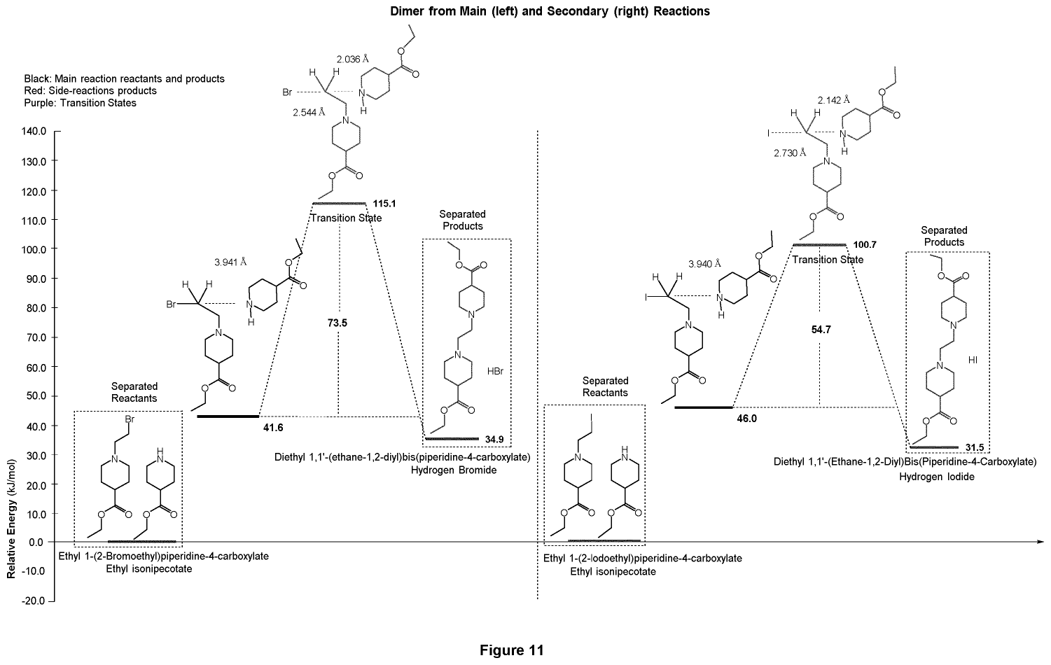

The reaction of ethyl isonipecotate with 1-bromo2-iodoethane was also studied at the B3LYP/3-21G level of theory. The energy barrier for the main reaction is comparable (78.1 kJ/mol) to that of the reference case (80.5 kJ/mol). The energy barrier for the secondary reaction (97.7 kJ/mol) is significantly less favorable than that of the reference case (66.6 kJ/mol), showing that the secondary reaction occurs to a lesser extent than that of the reference case. The formation of the dimer diethyl 1,1′-(Ethane-1,2-diyl)Bis(Piperidine-4-Carboxylate) through the main reaction product ethyl 1-(2-bromoethyl)piperidine-4-carboxylate and through the secondary product ethyl 1-(2-iodoethyl)piperidine-4-carboxylate is less energetically favorable (73.5 kJ/mol and 54.7 kJ/mol, respectively) than the reference case (43.7 kJ/mol and 73.5 kJ/mol, respectively). The results are presented in Table 5 and FIGS. 9, 10 and 11.

TABLE 5
Energy barriers and overall energy balances for 1-bromo-2-iodoethane reactions.

| | | Overall |
|---|---|---|
| | Energy | Energy |
| | Barrier | Balance |
| Chemical Reaction Scheme | (kJ/mol) | (kJ/mol) |
| 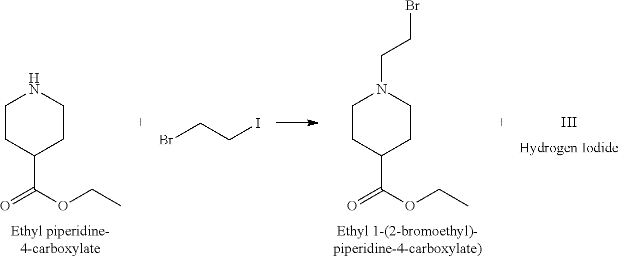 | 78.1 | −13.7 |
| 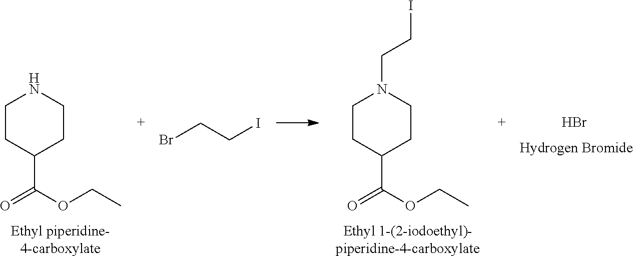 | 97.7 | 30.0 |
| 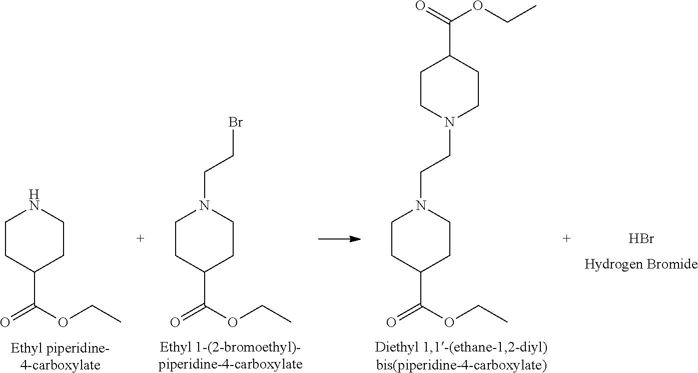 | 73.5 | 34.9 |
| 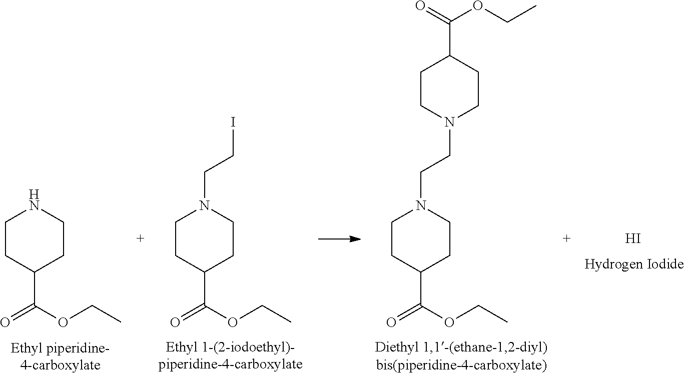 | 54.7 | 31.5 |

FIGS. 9, 10, and 11 show: Energy graphs for 1-bromo-2-iodoethane reactions.

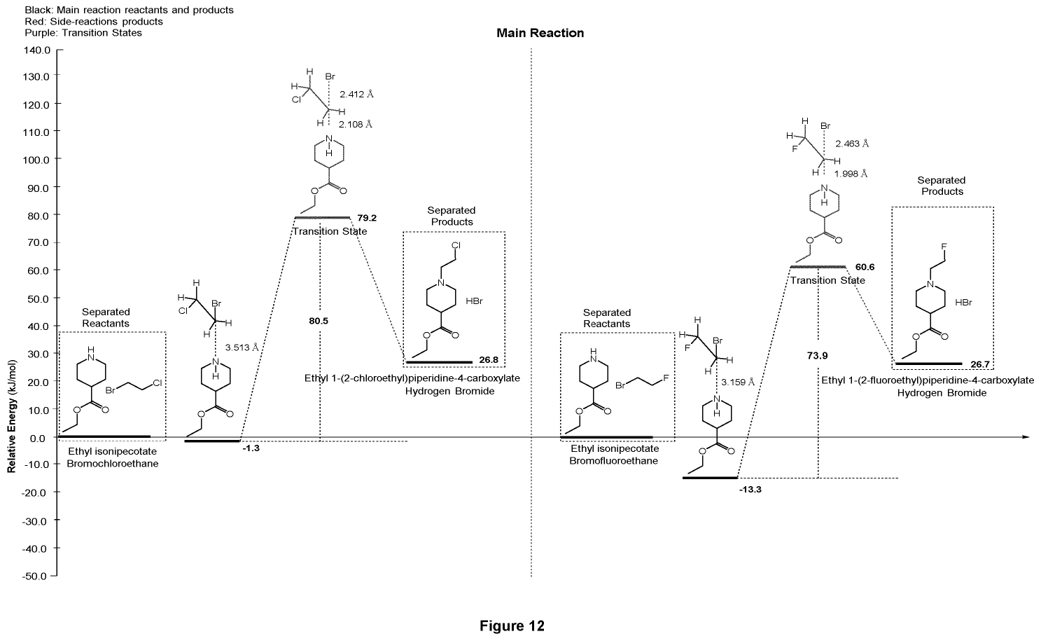

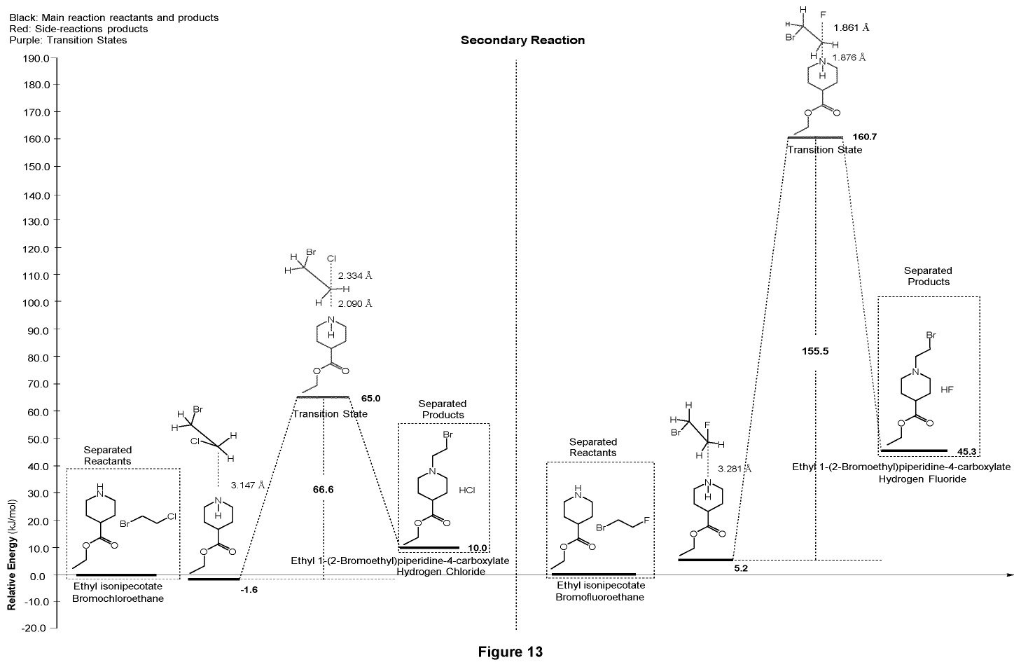

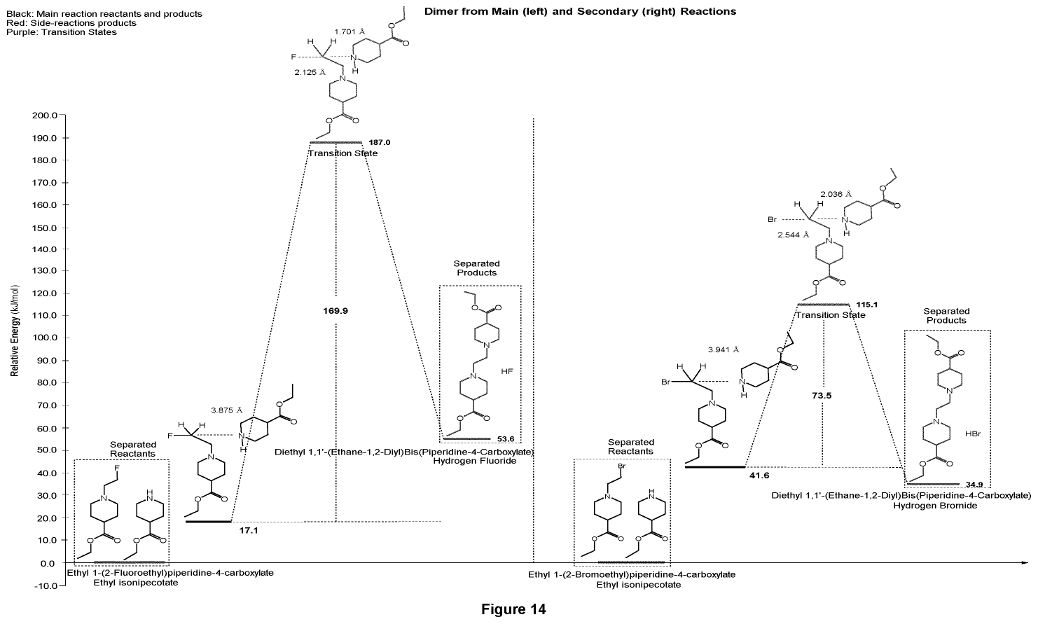

The reaction of ethyl isonipecotate with 1-bromo-2-fluoroethane presented similar results to those observed for 1-bromo-2-iodoethane. The energy barrier for the main reaction is slightly more favorable (73.9 kJ/mol) than the reference case (80.5 kJ/mol). The energy barrier for the secondary reaction (155.5 kJ/mol) is significantly less favorable than that of the reference case (66.6 kJ/mol), showing that the secondary reaction occurs to a lesser extent than that of the reference case. The formation of the dimer diethyl 1,1′-(ethane-1,2-diyl)bis(piperidine-4-carboxylate) through the main reaction product ethyl 1-(2-fluoroethyl)piperidine-4-carboxylate (169.9 kJ/mol) is less favorable than the reference case (73.5 kJ/mol). The formation of the dimer through the secondary product is the same reaction observed for the reference case. The results are presented in Table 6 and FIGS. 12, 13 and 14.

TABLE 6
Energy barriers and overall energy balances for1- bromo2-fluoroethane reactions.

| | | Overall |
|---|---|---|
| | Energy | Energy |
| | Barrier | Balance |
| Chemical Reaction Scheme | (kJ/mol) | (kJ/mol) |
| 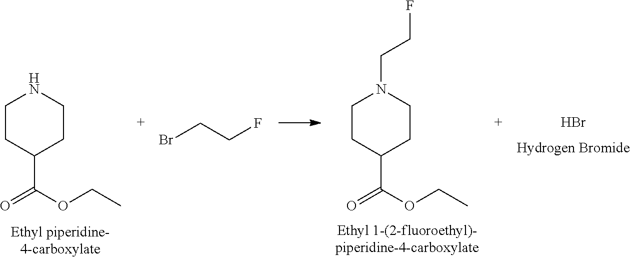 | 73.9 | 26.7 |
| 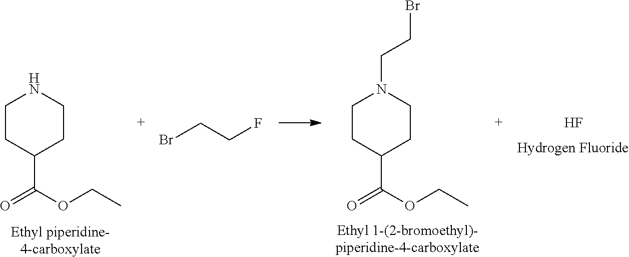 | 97.7 | 30.0 |
| 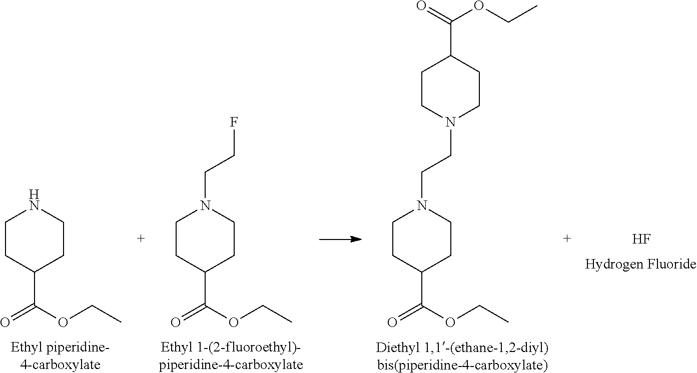 | 169.9 | 53.6 |
| 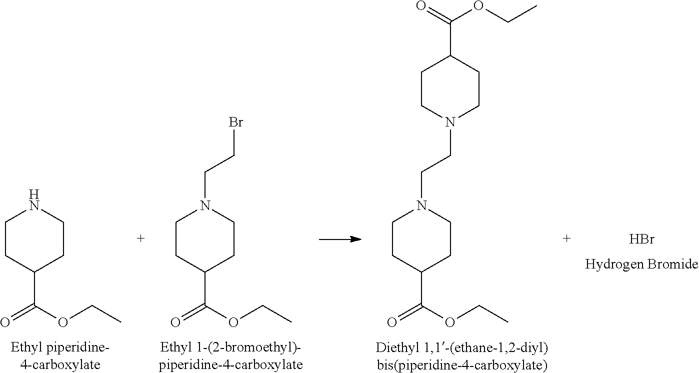 | 73.5 | 34.9 |

FIGS. 12, 13 and 14 show: Energy graphs for 1-bromo-2-fluoroethane reactions.

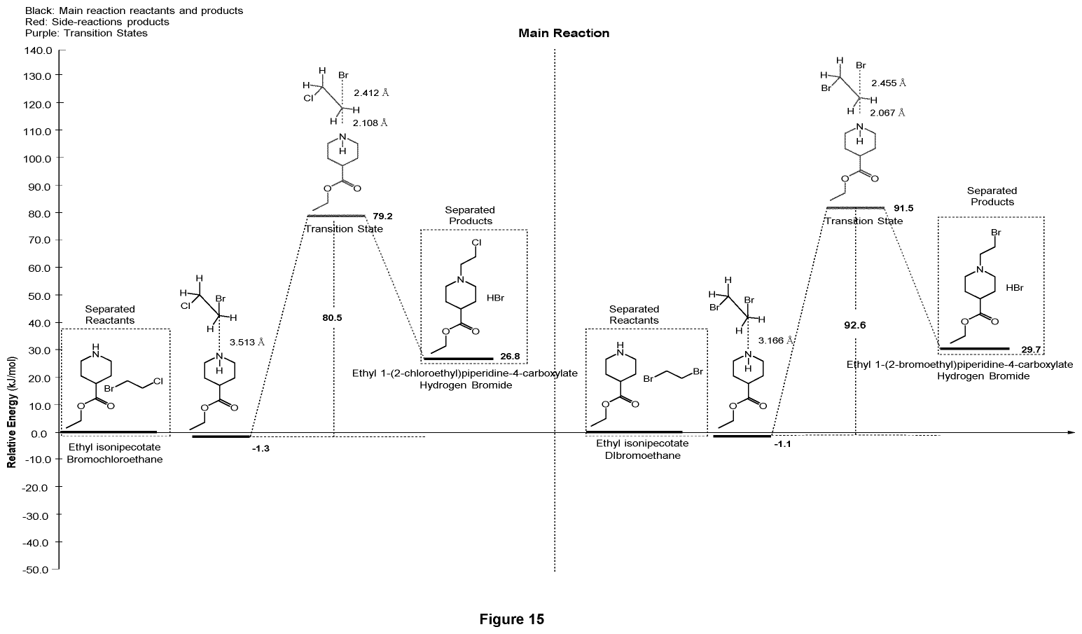

The energy barrier for the reaction of ethyl isonipecotate with 1,2-dibromoethane is a little higher (92.6 kJ/mol) than that of the reference case main reaction (80.5 kJ/mol), showing that a slight increase on the process temperature and/or a slightly longer residence time will give similar conversion to that of the reference case. With 1,2-dibromoethane there is no secondary reaction and the route for the formation of the dimer is the same as the one which may occur in the reference case through the secondary product. The results are presented in Table 6 and FIG. 15.

TABLE 7
Energy barriers and overall energy balances for 1,2-dibromoethane reactions.

| | | Overall |
|---|---|---|
| | Energy | Energy |
| | Barrier | Balance |
| Chemical Reaction Scheme | (kJ/mol) | (kJ/mol) |
| 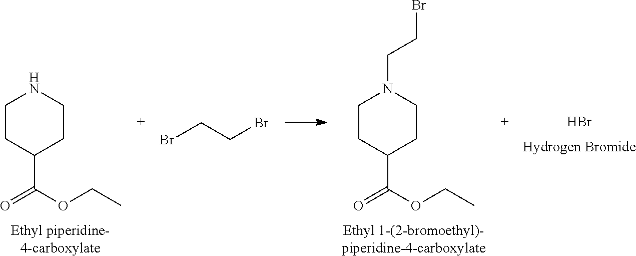 | 92.6 | 29.7 |
| 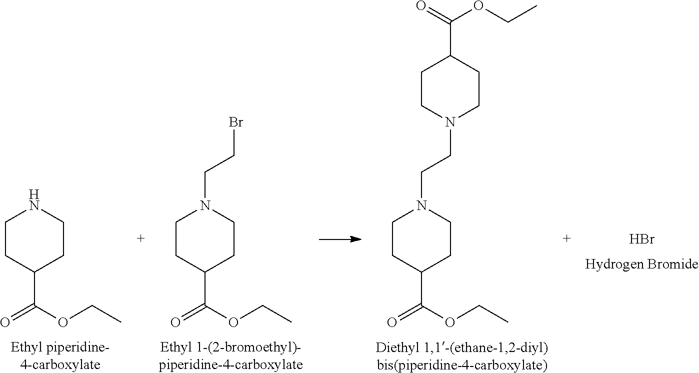 | 73.5 | 34.9 |

FIG. 15 shows: Energy graph for 1,2-dibromoethane reaction.

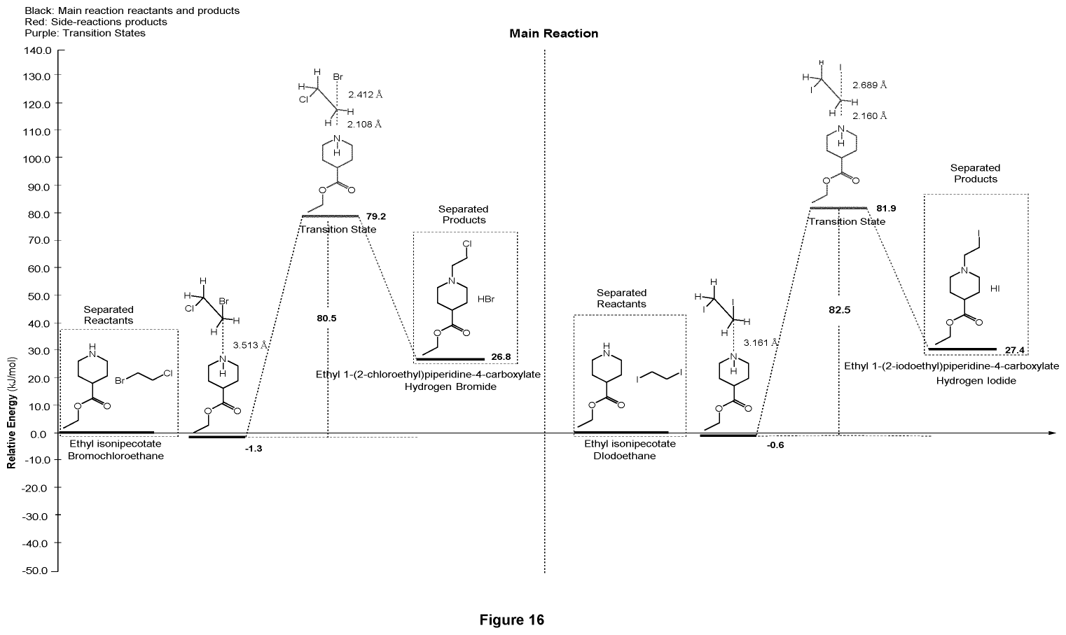

The energy barrier for the reaction of ethyl isonipecotate with 1,2-diiodoethane is similar (82.5 kJ/mol) to reference case main reaction (80.5 kJ/mol). No secondary reaction occurs and the reaction of formation of the dimer is the same as that of the secondary reaction of 1-bromo2-iodoethane (energy barrier 54.7 kJ/mol).The results are presented in Table 8 and FIG. 16.

TABLE 8
Energy barriers and overall energy balances for 1,2-diiodoethane reactions.

| | | Overall |
|---|---|---|
| | Energy | Energy |
| | Barrier | Balance |
| Chemical Reaction Scheme | (kJ/mol) | (kJ/mol) |
| 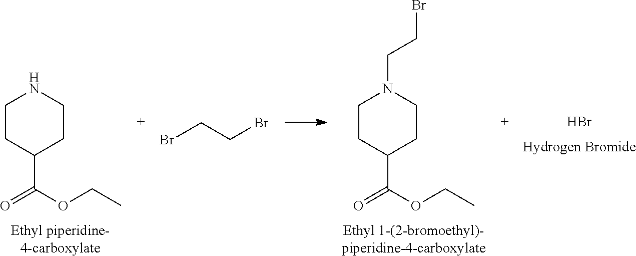 | 82.5 | 27.4 |
| 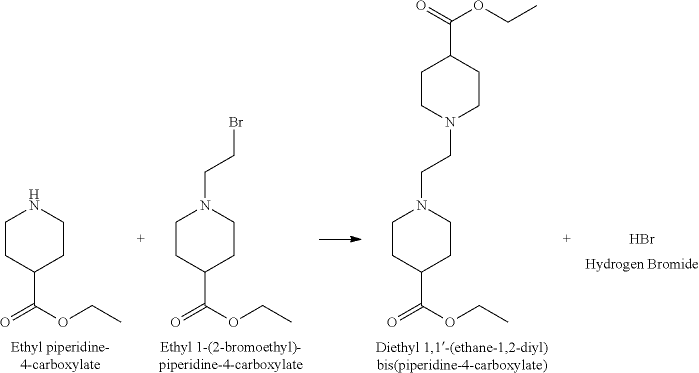 | 54.7 | 41.9 |

FIG. 16 shows: Energy graph for 1,2-diiodoethane reaction (right) and reference case (left).

The quantum mechanics calculations presented above together with the experimental results obtained for the reaction between ethyl isonipecotate and 1-bromo-2-chloroethane enable modeling of the reactions between pyrrolidine, piperidine and azepane and 1-bromo-2-chloroethane, respectively. Based on energy barriers comparison and the experimental results obtained on the synthesis of ethyl 1-(2-chloroethyl)piperidine-4-carboxylate, the synthesis in batch mode of 1-(2-chloroethyl)piperidine, 1-(2-chloroethyl)pyrrolidine and 1-(2-chloroethyl)azepane can be accomplished according to the experimental conditions described in example 1, with minor temperature adjustments e.g, by slightly decreasing the temperature in the cases where the energy barrier for the formation of the dimer impurity is lower than the corresponding energy barrier for the reference case described in example 1. The synthesis of these three compounds in flow mode, can be accomplished according to the conditions described in example 10, with minor residence times adjustments, e.g., by shortening the residence time in the cases where the energy barrier for the formation of the dimer impurity is lower than the corresponding energy barrier for the reference case described in example 10.

Additionally, the experimental results obtained for the reaction between ethyl isonipecotate and 1-bromo-2-chloroethane in combination with the energy barriers comparison for the reactions between ethyl isonipecotate and the alkylating reagents 1-bromo-2-iodoethane, 1-bromo-2-fluoroethane, 1,2-dibromoethane and 1,2-diiodoethane, showed that the synthesis of 1-(2-bromoethyl)piperidine-4-carboxylate by reaction of ethyl isonipecotate with 1-bromo-2-iodoethane or 1,2-dibromoethane, the synthesis of 1-(2-fluoroethyl)piperidine-4-carboxylate by reaction of ethyl isonipecotate with 1-bromo-2-fluoroethane and the synthesis of 1-(2-iodoethyl)piperidine-4-carboxylate by reaction of ethyl isonipecotate with 1,2-diiodoethane can be accomplished according to the conditions of example 1, in batch mode, and according to the conditions of example 10, in flow mode, or in batch mode, according to the experimental conditions described in example 1.

### EXAMPLES

The following examples are set forth to aid understanding of the invention but are not intended to, and should not be considered to, limit its scope in any way.

### Example 1

### Preparation of ethyl 1-(2-chloroethyl)piperidine-4-carboxylate

To 1-bromo-2-chloroethane (2.70 mL, 32.44 mmol, 10 equiv.) was added ethyl isonipecotate (0.5 mL, 3.24 mmol) followed by N,N-diisopropylethylamine (1.13 mL, 6.49 mmol). The reaction mixture was stirred for 12 hours at 24° C. and then diluted with n-heptane (3.0 mL). The suspension was filtered and extracted with water (1.5 mL). The organic layer was concentrated under vacuum resulting in the desired compound (yellowish oil, 0.65 g, 91.3%). The product was analyzed by GC resulting in 8.5% of respective dimeric side product.

### Example 2

### Preparation of ethyl 1-(2-chloroethyl)piperidine-4-carboxylate

To 1-bromo-2-chloroethane (2.70 mL, 32.44 mmol, 10 equiv.) was added ethyl isonipecotate (0.5 mL, 3.24 mmol) followed by triethylamine (0.91 mL, 6.49 mmol). The reaction mixture was stirred for 12 hours at 24° C. and then diluted with n-heptane (3.0 mL). The suspension was filtered and extracted with water (1.5 mL). The organic layer was concentrated under vacuum resulting in the desired compound (yellowish oil, 0.67 g, 94.4%). The product was analyzed by GC resulting in 8.1% of respective dimeric side product.

### Example 3

### Preparation of ethyl 1-(2-chloroethyl)piperidine-4-carboxylate

To 1-bromo-2-chloroethane (2.70 mL, 32.44 mmol, 10 equiv.) was added ethyl isonipecotate (0.5 mL, 3.24 mmol) followed by N-methylpyrrolidine (0.68 mL, 6.49 mmol). The reaction mixture was stirred for 12 hours at 24° C. and then diluted with n-heptane (3.0 mL). The suspension was filtered and extracted with water (1.5 mL). The organic layer was concentrated under vacuum resulting in the desired compound (colorless liquid, 0.27 g, 38.0%). The product was analyzed by GC resulting in 0.3% of respective dimeric side product.

### Example 4

### Preparation of 4-(2-chloroethyl)morpholine

To 1-bromo-2-chloroethane (4.76 mL, 57.19 mmol, 10 equiv.) was added morpholine (0.5 mL, 5.72 mmol) followed by N,N-diisopropylethylamine (1.99 mL, 11.44 mmol). The reaction mixture was stirred for 6 hours at 24° C. and then diluted with n-heptane (4.0 mL). The suspension was filtered and extracted with water (2.0 mL). The organic layer was concentrated under vacuum resulting in the desired compound (yellowish oil, 0.68 g, 79.6%). The product was analyzed by GC resulting in 1.5% of respective dimeric side product.

### Example 5

### Preparation of 4-benzyl-1-(2-chloroethyl)piperidine

To 1-bromo-2-chloroethane (2.37 mL, 28.47 mmol, 10 equiv.) was added 4-benzylpiperidine (0.5 mL, 2.85 mmol) followed by N,N-diisopropylethylamine (0.99 mL, 5.69 mmol). The reaction mixture was stirred for 12 hours at 24° C. and then diluted with n-heptane (3.0 mL). The suspension was filtered and extracted with water (1.5 mL). The organic layer was concentrated under vacuum resulting in the desired compound (white solid, 0.62 g, 92.0%). The product was analyzed by GC resulting in 21.8% of respective dimeric side product.

### Example 6

### Preparation of 1-(2-chloroethyl)-4-methylpiperazine

To 1-bromo-2-chloroethane (3.75 mL, 45.05 mmol, 10 equiv.) was added 1-methylpiperazine (0.5 mL, 4.51 mmol) followed by N,N-diisopropylethylamine (1.57 mL, 9.01 mmol). The reaction mixture was stirred for 6 hours at 24° C. and then diluted with n-heptane (4.0 mL). The suspension was filtered and extracted with water (2.0 mL). The organic layer was concentrated under vacuum resulting in the desired compound (white solid, 0.24 g, 33.1%). The product was analyzed by GC resulting in the absence of respective dimeric side product.

### Example 7

### Preparation of 1-(2-chloroethyl)-4-phenylpiperazine

To 1-bromo-2-chloroethane (2.72 mL, 32.68 mmol, 10 equiv.) was added 1-phenylpiperazine (0.5 mL, 3.27 mmol) followed by N,N-diisopropylethylamine (1.14 mL, 6.54 mmol). The reaction mixture was stirred for 48 hours at 24° C. and then diluted with n-heptane (4.0 mL). The suspension was filtered and extracted with water (2.0 mL). The organic layer was concentrated under vacuum resulting in the desired compound (yellowish solid, 0.36 g, 49.5%). The product was analyzed by GC resulting in 4.8% of respective dimeric side product.

### Example 8

### Preparation of 1-(2-chloroethyl)-4-methylpiperidine

To 1-bromo-2-chloroethane (3.52 mL, 42.29 mmol, 10 equiv.) was added 4-methylpiperidine (0.5 mL, 4.23 mmol) followed by N,N-diisopropylethylamine (1.47 mL, 8.46 mmol). The reaction mixture was stirred for 6 hours at 24° C. and then diluted with n-heptane (4.0 mL). The suspension was filtered and extracted with water (2.0 mL). The organic layer was concentrated under vacuum resulting in the desired compound (colorless liquid, 0.61 g, 89.9%). The product was analyzed by GC resulting in 23.1% of respective dimeric side product.

### Example 9

### Preparation of ethyl 1-(3-chloropropyl)piperidine-4-carboxylate

To 1-bromo-2-chloropropane (3.21 mL, 32.46 mmol, 10 equiv.) was added ethyl isonipecotate (0.5 mL, 3.25 mmol) followed by N,N-diisopropylethylamine (1.13 mL, 6.5 mmol). The reaction mixture was stirred for 6 hours at 24° C. and then diluted with n-heptane (4.0 mL). The suspension was filtered and extracted with water (2.0 mL). The organic layer was concentrated under vacuum resulting in the desired compound (yellowish oil, 0.66 g, 87.1%). The product was analyzed by GC resulting in 0.1% of respective dimeric side product.

### Example 10

### Preparation of ethyl 1-(2-chloroethyl)piperidine-4-carboxylate

In a plate microreactor (19.5 μL) 1-bromo-2-chloroethane (flowrate: 3.49 μL/min., 7 equiv.) was mixed with ethyl isonipecotate in N,N-diisopropylethylamine (0.92 M, flowrate: 3.01 μL/min.) at 100° C. The reaction was quenched and extracted with water. The organic layer was concentrated under vacuum resulting in the desired compound. The product was analyzed by GC resulting in 29% of unreacted ethyl isonipecotate and 11% of respective dimeric side product.

### Example 11

### Preparation of ethyl 1-(2-chloroethyl)piperidine-4-carboxylate

In a plate microreactor (10 μL) 1-bromo-2-chloroethane (flowrate: 1.33 μL/min., 9 equiv.) was mixed with ethyl isonipecotate in N,N-diisopropylethylamine (0.92 M, flowrate: 2.00 μL/min.) at 100° C. The reaction was quenched and extracted with water. The organic layer was concentrated under vacuum resulting in the desired compound. The product was analyzed by GC resulting in 35% of unreacted ethyl isonipecotate and 18% of respective dimeric side product.

### Example 12

### Preparation of ethyl 1-(2-chloroethyl)piperidine-4-carboxylate

In a PFA coil reactor (2.52 mL) 1-bromo-2-chloroethane (flowrate: 0.992 mL/min., 6 equiv.) was mixed with ethyl isonipecotate in 1,8-diazabicyclo[5.4.0]undec-7-ene (2.93 M, flowrate: 0.688 mL/min.) at 70° C. The reaction was quenched with water and extracted with n-heptane. The organic layer was concentrated under vacuum resulting in the desired compound (yellowish oil, 74%). The product was analyzed by GC resulting in 10.0% of respective dimeric side product.

### Example 13

### Preparation of ethyl 1-(2-chloroethyl)piperidine-4-carboxylate

In a PFA coil reactor (2.52 mL) 1-bromo-2-chloroethane (flowrate: 0.992 mL/min., 6 equiv.) was mixed with ethyl isonipecotate in 1,8-diazabicyclo[5.4.0]undec-7-ene (2.93 M, flowrate: 0.688 mL/min.) at 90° C. The reaction was quenched with water and extracted with n-heptane. The organic layer was concentrated under vacuum resulting in the desired compound (yellowish oil, 68%). The product was analyzed by GC resulting in 9.9% of respective dimeric side product.

### Example 14

### Preparation of ethyl 1-(2-chloroethyl)piperidine-4-carboxylate

In a PFA coil reactor (2.52 mL) 1-bromo-2-chloroethane (flowrate: 0.248 mL/min., 6 equiv.) was mixed with ethyl isonipecotate in 1,8-diazabicyclo[5.4.0]undec-7-ene (2.93 M, flowrate: 0.172 mL/min.) at 70° C. The reaction was quenched with water and extracted with n-heptane. The organic layer was concentrated under vacuum resulting in the desired compound (yellowish oil, 68%). The product was analyzed by GC resulting in 3.7% of respective dimeric side product.

### Example 15

### Preparation of ethyl 1-(2-chloroethyl)piperidine-4-carboxylate

In a PFA coil reactor (2.52 mL) 1-bromo-2-chloroethane (flowrate: 1.189 mL/min., 10 equiv.) was mixed with ethyl isonipecotate in 1,8-diazabicyclo[5.4.0]undec-7-ene (2.93 M, flowrate: 0.491 mL/min.) at 70° C. The reaction was quenched with water and extracted with n-heptane. The organic layer was concentrated under vacuum resulting in the desired compound (yellowish oil, 58%). The product was analyzed by GC resulting in 6.3% of respective dimeric side product.

### Example 16

### Preparation of 1-(2-chloroethyl)pyrrolidine

In a plate microreactor (19.5 μL) 1-bromo-2-chloroethane (flowrate: 2.27 μL/min.7 equiv.) is mixed with pyrrolidine in N,N-diisopropylethylamine (0.92 M, flowrate: 4.23 μL/min.) at 100° C. The reaction is quenched and extracted with water. The organic layer is concentrated under vacuum resulting in the desired compound.

Alternatively, in a PFA coil (2.52 mL) 1-bromo-2-chloroethane (flowrate: 1.189 mL/min., 10 equiv.) is mixed with pyrrolidine in 1,8-diazabicyclo[5.4.0]undec-7-ene (2.93 M, flowrate: 0.491 mL/min.) at 70° C. The reaction is quenched and extracted with water. The organic layer is concentrated under vacuum resulting in the desired compound.

### Example 17

### Preparation of 1-(2-chloroethyl)pyrrolidine

Add pyrrolidine (0.3 mL, 3.7 mmol) to 1-bromo-2-chloroethane (2.70 mL, 32.44 mmol, 9 equiv.) followed by N,N-diisopropylethylamine (1.13 mL, 6.49 mmol). The reaction mixture stirs for 12 hours at 24° C. and then is diluted with n-heptane (3.0 mL). The suspension is filtered and extracted with water (1.5 mL). The organic layer is concentrated under vacuum to yield the desired compound.

### Example 18

### Preparation of 1-(2-chloroethyl)piperidine

In a plate microreactor (19.5 μL) 1-bromo-2-chloroethane (flowrate: 2.27 μL/min., 7 equiv.) is mixed with piperidine in N,N-diisopropylethylamine (0.92 M, flowrate: 4.23 μL/min.) at 100° C. The reaction is quenched and extracted with water. The organic layer is concentrated under vacuum resulting in the desired compound.

Alternatively, in a PFA coil (2.52 mL) 1-bromo-2-chloroethane (flowrate: 1.189 mL/min., 10 equiv.) is mixed with piperidine in 1,8-diazabicyclo[5.4.0]undec-7-ene (2.93 M, flowrate: 0.491 mL/min.) at 70° C. The reaction is quenched and extracted with water. The organic layer is concentrated under vacuum resulting in the desired compound.

### Example 19

### Preparation of 1-(2-chloroethyl)piperidine

Add piperidine (0.3 mL, 3.0 mmol) to 1-bromo-2-chloroethane (2.7 mL, 32.44 mmol, 11 equiv.) followed by N,N-diisopropylethylamine (1.13 mL, 6.49 mmol). The reaction mixture stirs for 12 hours at 24° C. and then is diluted with n-heptane (3.0 mL). The suspension is filtered and extracted with water (1.5 mL). The organic layer is concentrated under vacuum to yield the desired compound.

### Example 20

### Preparation of 1-(2-chloroethyl)azepane

In a plate microreactor (19.5 μL) 1-bromo-2-chloroethane (flowrate: 2.27 μL/min., 7 equiv.) is mixed with azepane in N,N-diisopropylethylamine (0.92 M, flowrate: 4.23 μL/min.) at 100° C. The reaction is quenched and extracted with water. The organic layer is concentrated under vacuum resulting in the desired compound.

Alternatively, in a PFA coil (2.52 mL) 1-bromo-2-chloroethane (flowrate: 1.189 mL/min., 10 equiv.) is mixed with azepane in 1,8-diazabicyclo[5.4.0]undec-7-ene (2.93 M, flowrate: 0.491 mL/min.) at 70° C. The reaction is quenched and extracted with water. The organic layer is concentrated under vacuum resulting in the desired compound.

### Example 21

### Preparation of 1-(2-chloroethyl)azepane

Add azepane (0.3 mL, 2.7 mmol) to 1-bromo-2-chloroethane (2.7 mL, 36.24 mmol, 13 equiv.) followed by N,N-diisopropylethylamine (1.13 mL, 6.49 mmol). The reaction mixture stirs for 12 hours at 24° C. and then is diluted with n-heptane (3.0 mL). The suspension is filtered and extracted with water (1.5 mL). The organic layer is concentrated under vacuum to yield the desired compound.

### Example 22

### Preparation of ethyl 1-(2-bromoethyl)-piperidine-4-carboxylate

In a plate microreactor (19.5 μL) previously melted 1-bromo-2-iodoethane (flowrate: 2.27 μL/min, 7 equiv.) is mixed with ethyl isonipecotate in N,N-diisopropylethylamine (0.92 M, flowrate: 4.23 μL/min.) at 100° C. The reaction is quenched and extracted with water. The organic layer is concentrated under vacuum resulting in the desired compound.

Alternatively, in a PFA coil (2.52 mL) 1-bromo-2-chloroethane (flowrate: 1.189 mL/min., 10 equiv.) is mixed azepane in 1,8-diazabicyclo[5.4.0]undec-7-ene (2.93 M, flowrate: 0.491 mL/min.) at 70° C. The reaction is quenched and extracted with water. The organic layer is concentrated under vacuum resulting in the desired compound.

### Example 23

### Preparation of ethyl 1-(2-bromoethyl)-piperidine-4-carboxylate

Add ethyl isonipecotate (0.6 mL, 3.8 mmol) to previously melted 1-bromo-2-iodoethane (7.6 g, 32.44 mmol, 9 equiv.) followed by N,N-diisopropylethylamine (1.13 mL, 6.49 mmol). The reaction mixture stirs for 12 hours at 24° C. and then is diluted with n-heptane (3.0 mL). The suspension is filtered and extracted with water (1.5 mL). The organic layer is concentrated under vacuum to yield the desired compound.

### Example 24

### Preparation of ethyl 1-(2-bromoethyl)-piperidine-4-carboxylate

In a plate microreactor (19.5 μL) 1,2-dibromoethane (flowrate: 2.27 μL/min, 7 equiv.) is mixed with ethyl isonipecotate in N,N-diisopropylethylamine (0.92 M, flowrate: 4.23 μL/min.) at 100° C. The reaction is quenched and extracted with water. The organic layer is concentrated under vacuum resulting in the desired compound.

### Example 25

### Preparation of ethyl 1-(2-bromoethyl)-piperidine-4-carboxylate

Add ethyl isonipecotate (0.6 mL, 3.8 mmol) to 1,2-dibromoethane (3.0 ml, 32.65 mmol, 9 equiv.) followed by N,N-diisopropylethylamine (1.13 mL, 6.49 mmol). The reaction mixture stirs for 12 hours at 24° C. and then is diluted with n-heptane (3.0 mL). The suspension is filtered and extracted with water (1.5 mL). The organic layer is concentrated under vacuum to yield the desired compound.

### Example 26

### Preparation of ethyl 1-(2-fluoroethyl)-piperidine-4-carboxylate

In a plate microreactor (19.5 μL) 1-bromo-2-fluoroethane (flowrate: 2.27 μL/min, 8 equiv.) is mixed with ethyl isonipecotate in N,N-diisopropylethylamine (0.92 M, flowrate: 4.23 μL/min.) at 100° C. The reaction is quenched and extracted with water. The organic layer is concentrated under vacuum resulting in the desired compound.

### Example 27

### Preparation of ethyl 1-(2-fluoroethyl)-piperidine-4-carboxylate

Add ethyl isonipecotate (0.6 mL, 3.8 mmol) to 1-bromo-2-fluoroethane (2.7 ml, 36.2 mmol, 10 equiv.) followed by N,N-diisopropylethylamine (1.13 mL, 6.49 mmol). The reaction mixture stirs for 12 hours at 24° C. and then is diluted with n-heptane (3.0 mL). The suspension is filtered and extracted with water (1.5 mL). The organic layer is concentrated under vacuum to yield the desired compound.

### Example 28

### Preparation of ethyl 1-(2-iodoethyl)-piperidine-4-carboxylate

In a plate microreactor (19.5 μL) 1,2-diodoethane (flowrate: 2.50 μL/min, 5 equiv.) is mixed with ethyl isonipecotate in N,N-diisopropylethylamine (0.92 M, flowrate: 4.23 μL/min.) at 100° C. The reaction is quenched and extracted with water. The organic layer is concentrated under vacuum resulting in the desired compound.

### Example 29

### Preparation of ethyl 1-(2-iodoethyl)-piperidine 4-carboxylate

Add ethyl isonipecotate (0.3 mL, 1.9 mmol) to 1,2-diodoethane (4.3 ml, 32.44 mmol) followed by N,N-diisopropylethylamine (1.13 mL, 6.49 mmol). The reaction mixture stirs for 12 hours at 24° C. and then is diluted with n-heptane (3.0 mL). The suspension is filtered and extracted with water (1.5 mL). The organic layer is concentrated under vacuum to yield the desired compound.

1-22. (canceled)

23. A process for the preparation of a compound of formula II: 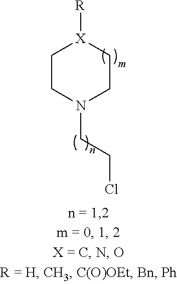
where Et=ethyl; Bn=benzyl and Ph=phenyl; and wherein where X=O, R is absent;
comprising the step of reacting a cyclic amine with a bifunctional alkylating agent to form a compound of formula II, wherein the said above step is solvent-free, wherein the cyclic amine is a compound of formula I or a salt thereof: 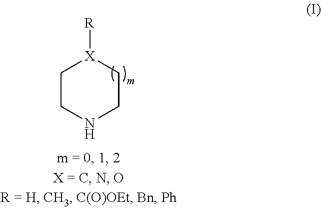
where Et=ethyl; Bn=benzyl and Ph=phenyl; and
wherein the process further comprises the presence of a further organic base; and
wherein solvent-free means no solvent is specifically added to perform the reaction step and the cyclic amine, the alkylating agent and the organic base, are not encompassed by the term “solvent”.

24. A process according to claim 23, wherein the process is carried out in a single reaction step.

25. A process according to claim 23, wherein the alkylating agent is used in an excess of from 5 to 15 molar equivalents compared to the cyclic amine compound.

26. A process according to claim 23, wherein the cyclic amine is reacted with 1-bromo-2-chloroethane or 1-bromo-3-chloropropane in the presence of an organic base to form a 1-chloroethyl or a 1-chloropropyl N-substituted cyclic amine.

27. A process according to claim 23, wherein the process is carried out in batch mode.

28. A process according to claim 23, wherein the process is carried out in continuous mode.

29. A process according to claim 28, wherein the process is carried out as a continuous flow procedure.

30. A process according to claim 23, wherein the further organic base is selected from the group consisting of N,N-diisopropylethylamine, triethylamine, tributylamine, N-methylimidazole, 4-(dimethylamino)pyridine, 1,8-diazabicyclo[5.4.0]undec-7-ene, and 1,5-diazabicyclo[4.3.0]non-5-ene.

31. A process according to claim 30, wherein for a process carried out in batch mode the further organic base is N,N-diisopropylethylamine or triethylamine.

32. A process according to claim 30, wherein for a process carried out in continuous mode the further organic base is 1,8-diazabicyclo[5.4.0]undec-7-ene or 1,5-diazabicyclo[4.3.0]non-5-ene.

33. A process according to claim 23, wherein the cyclic amine is a 6-membered cyclic amine.

34. A process according to claim 33, wherein the 6-membered cyclic amine is 4-methylpiperidine, ethyl 4-piperidinecarboxylate, 1-methylpiperazine, 1-phenylpiperazine, 1-benzylpiperazine, morpholine, pyrrolidine or hexamethyleneimine.

35. A process according to claim 23 provided the process is carried out in batch mode, wherein the temperature is between about 20° C. and about 100° C.

36. A process according to claim 23 provided the process is carried out in continuous mode, wherein the temperature is between about 60° C. and about 100° C.

37. A process according to claim 23 provided the process is carried out in batch mode, wherein the reaction time is between about 6 hours and about 48 hours.

38. A process according to claim 23 provided the process is carried out in continuous mode, wherein the reaction time is between about 2 min and about 20 min.

39. A process according to claim 23, wherein the compound of formula II is 1-(2-chloroethyl)-4-methylpiperidine, 1-(3-chloropropyl)-4-methylpiperidine, ethyl 1-(2-chloroethyl)piperidine-4-carboxylate, ethyl 1-(3-chloropropyl)piperidine-4-carboxylate, 1-(2-chloroethyl)-4-methylpiperazine, 1-(3-chloropropyl)-4-methylpiperazine, 1-(2-chloroethyl)-4-phenylpiperazine, 1-(3-chloropropyl)-4-phenylpiperazine, 1-(2-chloroethyl)-4-benzylpiperazine, 1-(3-chloropropyl)-4-benzylpiperazine, 1-(2-chloroethyl)morpholine, 1-(3-chloropropyl)morpholine, 1-(2-chloroethyl)pyrrolidine, 1-(3-chloropropyl)pyrrolidine, 1-(2-chloroethyl)hexamethyleneimine or 1-(3-chloropropyl)hexamethyleneimine.

40. A method comprising utilizing the chloroalkyl N-substituted cyclic amines of formula II 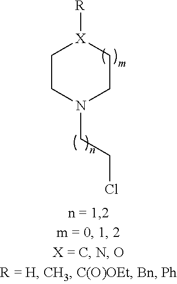
where Et=ethyl; Bn=benzyl and Ph=phenyl; and wherein where X=O, R is absent;
obtained according to the process of claim 23 in the preparation of active pharmaceutical ingredients or intermediates therefore comprising these moieties in their molecular structure.

41. The method according to claim 40, wherein the active pharmaceutical ingredient is umeclidinium bromide, ziprasidone, risperidone, trifluoperazine, trazodone, gefitinib, doxapram, domperidone, cetiedil, nabazenil, setastine, fedratinib or pitolisant.

42. A method comprising utilizing the chloroalkyl N-substituted cyclic amines of formula II: 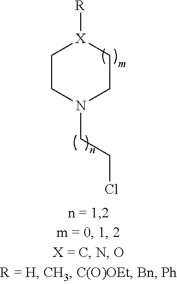
where Et=ethyl; Bn=benzyl and Ph=phenyl; and wherein where X=O, R is absent;
obtained according to the process of claim 23 in the preparation of agrochemical compounds or intermediates therefore comprising these moieties in their molecular structure.
